(19) United States
(12) Patent Application Publication
(10) Pub. No.: US 2013/0340110 A1
(43) Pub. Date: Dec. 19, 2013

(54) METHODS FOR SELECTION OF INTROGRESSION MARKER PANELS

(71) Applicant: AGRIGENETICS, INC., Indianapolis, IN (US)

(72) Inventors: KELLY R. ROBBINS, INDIANAPOLIS, IN (US); JAN ERIK BACKLUND, INDIANAPOLIS, IN (US)

(21) Appl. No.: 13/916,648

(22) Filed: Jun. 13, 2013

Related U.S. Application Data
(60) Provisional application No. 61/660,055, filed on Jun. 15, 2012.

(51) Int. Cl. A01H 1/04

(52) U.S. Cl. CPC A01H 1/04
USPC 800/260; 506/8; 506/17; 506/9

(57) ABSTRACT

This disclosure concerns marker-assisted plant selection and breeding. In specific embodiments, methods of identifying optimized marker panels for predicting the presence of a plant trait of interest, and/or marker panels thereby identified, are provided.

### CROSS-REFERENCE TO RELATED APPLICATION

This application claims the benefit of U.S. Provisional Application Ser. No. 61/660,055, filed Jun. 15, 2012, the disclosure of which is hereby incorporated by reference in its entirety, including all figures and tables.

### FIELD OF THE DISCLOSURE

The present disclosure relates to plant breeding. More specifically, the disclosure relates to the use of an improved system for the identification and selection of a set of plant genetic markers that are highly useful for the introgression of a trait of interest.

### BACKGROUND

The development of hybrid plant breeding has made possible considerable advances in quality and quantity of crops produced. Increased yield and the combination of desirable characteristics, such as resistance to disease and insects, heat and drought tolerance, and variations in plant composition are all possible, in part, due to plant hybridization procedures. Hybridization procedures rely on the contribution of pollen from a male parent plant to a female parent plant to produce the resulting hybrid.

The development of maize hybrids requires the development of homozygous inbred lines, the crossing of these lines, and the evaluation of the crosses. Pedigree breeding and recurrent selection are two breeding methods used to develop inbred lines from populations. Breeding programs combine desirable traits from two or more inbred lines, or various broad-based sources, into breeding pools from which new inbred lines are developed by selfing and selection of desired phenotypes. A hybrid maize variety is the cross of two such inbred lines, each of which may have one or more desirable characteristics absent in one of the inbred lines, or complementing characteristic(s) of the other. New inbred plants are crossed with other inbred lines, and the hybrids from these crosses are evaluated to determine which are desirable. Hybrid progeny of the first generation are designated F₁. The F₁ hybrid is typically more vigorous than its inbred parents. This hybrid vigor, termed heterosis, typically leads to, for example, increased vegetative growth and increased yield. Thus, in the development of hybrids, only the F₁ hybrids are generally sought.

To facilitate marker-assisted introgression, highly-informative markers (e.g., SNP markers that are polymorphic between recurrent and donor parents) are desirable across populations targeted for trait introgression. The identification of a subset of informative markers can be viewed as a combinatorial problem. The solution is theoretically simple to achieve, requiring the evaluation of every possible combination, but computationally unfeasible. For example, current trait introgression applications using a marker set of 256 markers would require a practitioner to evaluate 1.2×10²⁷⁹ marker combinations to exhaustively search all possible combinations, and this number increases exponentially as more markers are included in the marker set.

An ant colony algorithm (ACA) is a swarm intelligence algorithm that mimics the mechanism by which real ant colonies communicate in search of the best route to a food source. See, e.g., Dorigio et al. (1999) Artificial Life 5(2):137-72. In nature, ants deposit chemical pheromones on the ground to form paths for other ants to follow. Initially, ants will disperse randomly from the nest in search of food, returning after a food source has been found. Ants finding the fastest route to a food source traverse the distance between the nest and the found food source at a faster rate, depositing more pheromone in the process. As the pheromone level builds, more ants preferentially choose the shorter path over longer paths with less pheromone, thereby depositing even more pheromone in the process. According to the foregoing, the natural biological behavior of ant colonies describes the fundamental elements of a positive feedback system, whereby all the ants of the colony will eventually select the best route from the nest to the food source.

Ant colony optimization (ACO) of solutions to problems with large sample spaces has been shown to be an efficient technique in communications network routing (Dorigio et al. (1999), supra; disease identification (Ressom et al. (2007) Bioinformatics 23(5):619-26); disease classification (Robbins et al. (2007) Math. Med. Biol. 24:413-26; and livestock genotyping (Spangler et al. (2009) Anim. Genet. 40:308-14).

### BRIEF SUMMARY OF THE DISCLOSURE

Described herein are systems and methods for determining a set of genetic markers for use in plant breeding, the method comprising vectors, each storing one possible solution or “path”. The method models communication between these vectors, called “ants”, through an adaptive probability density function (PDF) referred to as a pheromone function. In embodiments, ants use the function to select subsets of genetic markers from the genome of a plant species of interest. The subsets of genetic markers may then be evaluated utilizing genetic information provided from a plurality of plants of interest. Based on the performance of the selected subset, the pheromone function may be updated, such that features yielding desirable solutions are more likely to be selected by ants in future iterations.

In particular embodiments, the ACA is adapted to identify a marker panel yielding optimal genome analysis coverage. In particular embodiments, the ACA is adapted to identify a marker panel yielding optimal linkage disequilibrium coverage.

In some embodiments, a TaqMan® SNP genotyping system (e.g., an OpenArray® genotyping system) is used to provide genetic information from a plant of interest.

The foregoing and other features will become more apparent from the following detailed description of several embodiments, which proceeds with reference to the accompanying figures.

### BRIEF DESCRIPTION OF THE FIGURES

FIGS. 1a-y include programming code that may be implemented on a computer to perform ant colony optimization according to particular embodiments.

FIGS. 2-3 include comparisons of the performance of marker panels resulting from several optimization methods: for “ACA,” sampling was based on the adaptive pheromone function; for “PS,” sampling was based on the proportion of times a marker was informative (polymorphic between donor and recurrent parent) across all parent combinations, and for “RS,” sampling was completely random. Performance (GA in FIG. 1; LD in FIG. 2) is expressed as the ratio of the coverage achieved by the selected marker panel and the coverage achieved when all markers are used (in this example, there were a total of 1371 markers). For each method, 24,000 marker subsets were evaluated, with only the top performing subset being presented.

FIG. 2 includes plots of GA coverage for ACA, PS, and RS. GA coverage is presented as the proportion of coverage retained in the selected panel as compared to the coverage obtained when using all available markers.

FIG. 3 includes plots of LD coverage for ACA, PS, and RS. LD coverage is presented as the proportion of coverage retained in the selected panel as compared to the coverage obtained when using all available markers.

FIG. 4 includes plots of informative marker positions for the 256 markers selected by ACA (ACA Markers), and all available markers (All Markers). FIG. 4a includes a plot of marker positions for D020083/SLB01. FIG. 4b includes a plot of marker positions for SLD25BM/SLB01.

### DETAILED DESCRIPTION

### I. Overview of Several Embodiments

With the development of thousands of genetic markers (e.g., SNP markers) for major crop species, panels of markers that will be effective across multiple crop lines for use in marker-assisted introgression of traits of interest (e.g., agronomically important traits) now theoretically exist. Identification of such effective panels will allow for further automation and increased efficiency of the introgression process. However, given the prohibitively large and growing number of available markers associated with panel selection for plant breeding projects, exhaustive evaluation of all possible marker panels is computationally unfeasible. As such, systems and methods are provided herein to efficiently search an expansive sample space of markers to find an optimal solution in the form of an optimized marker panel. In some embodiments, systems and methods are provided that can identify informative marker subsets while searching only a small fraction of an immense sample marker space.

An ant colony optimization (ACO) system utilizes a positive feedback communication system that mimics the pheromone trails used by real ant colonies to find the best route to a food source, in order to efficiently search prohibitively large sample spaces for optimal solutions. In embodiments of the invention, an ACO system may be adapted to identify a panel of known and/or empirically determinable genetic markers that may yield optimized genome analysis (GA) and/or linkage drag (LD) coverage for use in marker-assisted plant breeding (e.g., marker-assisted trait introgression). Using methods according to some embodiments of the invention, an ACO system is surprisingly effective at performing the task of identifying optimized marker panels for use in marker-assisted plant breeding, such that an ACO system consistently outperforms other optimization methods. The surprising superiority of methods according to particular embodiments may increase as the marker sample space increases.

In certain examples, an ACO was applied to identify a highly informative panel of 256 markers for plant breeding program development from a set of 1371 available SNP markers. In an application to 72 potential introgression projects, methods utilizing an ACO consistently outperformed all other tested methods. Using the identified set of 256 markers, 80% of the genome (GA) and LD coverage obtained when using all 1371 available markers was retained by the marker subset, further and quantitatively demonstrating the effectiveness of the methods utilizing an ACO.

### II. Abbreviations

ACA ant colony algorithm

ACO ant colony optimization

AFLP amplified fragment length polymorphism

DAF DNA amplification fingerprinting

GA genome analysis

LD linkage drag

PCR polymerase chain reaction

PDF probability density function

PS sampling based on prior information

QTL quantitative trait locus

RAPD random amplification of polymorphic DNA

RFLP restriction fragment length polymorphism

RS random sampling

SCAR sequence characterized amplified region

SNP single nucleotide polymorphism

### III. Terms

Ant: As used herein, an “ant” or “artificial ant” refers to an agent that moves from point to point. An “ant colony optimization” (ACO) system refers to a metaheuristic used for discrete, combinatorial optimization in some embodiments. In an ACO system, an ant may choose a next point to move to using a probabilistic function, both of trail accumulated on edges and of a heuristic value, which may be a function of the edge length. Ants will preferentially select discrete states with higher joint probabilities according to the pheromone function.

Backcrossing: Backcrossing methods may be used to introduce a nucleic acid sequence into plants. The backcrossing technique has been widely used for decades to introduce new traits into plants. Jensen, N., Ed. Plant Breeding Methodology, John Wiley & Sons, Inc., 1988. In a typical backcross protocol, the original variety of interest (recurrent parent) is crossed to a second variety (non-recurrent parent) that carries a gene of interest to be transferred. The resulting progeny from this cross are then crossed again to the recurrent parent, and the process is repeated until a plant is obtained wherein essentially all of the desired morphological and physiological characteristics of the recurrent plant are recovered in the converted plant, in addition to the transferred gene from the non-recurrent parent.

Genome analysis: “Genome analysis” generally refers to techniques that determine and compare genetic sequences. This includes DNA sequencing, routine use of DNA microarray technology for the analysis of gene expression profiles at the mRNA level and improved informatic tools to organize and analyze such data.

Isolated: An “isolated” biological component (such as a nucleic acid or protein) has been substantially separated, produced apart from, or purified away from other biological components in the cell of the organism in which the component naturally occurs (i.e., other chromosomal and extra-chromosomal DNA and RNA, and proteins), while effecting a chemical or functional change in the component (e.g., a nucleic acid may be isolated from a chromosome by breaking chemical bonds connecting the nucleic acid to the remaining DNA in the chromosome). Nucleic acid molecules and proteins that have been “isolated” include nucleic acid molecules and proteins purified by standard purification methods. The term also embraces nucleic acids and proteins prepared by recombinant expression in a host cell, as well as chemically-synthesized nucleic acid molecules, proteins, and peptides.

Linkage drag: “Linkage drag” refers to the length of donor genome segment surrounding a gene of introgression. The linkage drag segment is important as it may incorporate other less favourable alleles and drag them into the commercial population and the risk of this is related to its length. Molecular makers offer a tool in which the amount of wild or alien DNA can be monitored during each backcross generation.

Nucleic acid molecule: As used herein, the term “nucleic acid molecule” may refer to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A nucleic acid molecule as used herein is synonymous with “nucleic acid” and “polynucleotide.” The term, nucleic acid molecule, includes single- and double-stranded forms of DNA. A nucleic acid molecule can include either or both of naturally occurring and modified nucleotides linked together by naturally occurring and non-naturally occurring nucleotide linkages. Nucleic acid molecules may be modified chemically or biochemically, or may contain non-natural or derivatized nucleotide bases, as will be readily appreciated by those of skill in the art. The term “nucleic acid molecule” also includes any topological conformation, including single-stranded, double-stranded, partially duplexed, triplexed, hairpinned, circular, and padlocked conformations.

Locus: As used herein, the term “locus” refers to a position on the genome that corresponds to a measurable characteristic (e.g., a trait). An SNP locus is defined by a probe that hybridizes to DNA contained within the locus.

Marker: As used herein, a marker refers to a gene or nucleotide sequence that can be used to identify plants that are likely to have a particular allele and/or exhibit a particular trait or phenotype. A marker may be described as a variation at a given genomic locus. A genetic marker may be a short DNA sequence, such as a sequence surrounding a single base-pair change (single nucleotide polymorphism, or “SNP”), or a long sequence, for example, a minisatellite/simple sequence repeat (“SSR”). A “marker allele” refers to the version of the marker that is present in a particular plant. The term marker as used herein may refer to a cloned segment of plant chromosomal DNA, and may also or alternatively refer to a DNA molecule that is complementary to a cloned segment of plant chromosomal DNA.

In some embodiments, the presence of a marker in a plant may be detected through the use of a nucleic acid probe. A probe may be a DNA molecule or an RNA molecule. RNA probes can be synthesized by means known in the art, for example, using a DNA molecule template. A probe may contain all or a portion of the nucleotide sequence of the marker and additional, contiguous nucleotide sequence from the plant genome. This is referred to herein as a “contiguous probe.” The additional, contiguous nucleotide sequence is referred to as “upstream” or “downstream” of the original marker, depending on whether the contiguous nucleotide sequence from the plant chromosome is on the 5′ or the 3′ side of the original marker, as conventionally understood. As is recognized by those of ordinary skill in the art, the process of obtaining additional, contiguous nucleotide sequence for inclusion in a marker may be repeated nearly indefinitely (limited only by the length of the chromosome), thereby identifying additional markers along the chromosome. Any and all of the above-described varieties of markers may be used in some embodiments of the present invention.

An oligonucleotide probe sequence may be prepared synthetically or by cloning. Suitable cloning vectors are well-known to those of skill in the art. An oligonucleotide probe may be labeled or unlabeled. A wide variety of techniques exist for labeling nucleic acid molecules, including, for example and without limitation: Radiolabeling by nick translation; random priming; tailing with terminal deoxytransferase; etc., where the nucleotides employed are labeled, for example, with radioactive ³²P. Other labels which may be used include, for example and without limitation: Fluorophores; enzymes; enzyme substrates; enzyme cofactors; enzyme inhibitors; etc. Alternatively, the use of a label that provides a detectable signal, by itself or in conjunction with other reactive agents, may be replaced by ligands to which receptors bind, where the receptors are labeled (for example, by the above-indicated labels) to provide detectable signals, either by themselves, or in conjunction with other reagents. See, e.g., Leary et al. (1983) Proc. Natl. Acad. Sci. USA 80:4045-9.

A probe may contain a nucleotide sequence that is not contiguous to that of the original marker; this probe is referred to herein as a “noncontiguous probe.” The sequence of the noncontiguous probe is located sufficiently close to the sequence of the original marker on the chromosome so that the noncontiguous probe is genetically linked to the same marker or gene as the original marker. For example, in some embodiments, a noncontiguous probe can be located within 500 kb; 450 kb; 400 kb; 350 kb; 300 kb; 250 kb; 200 kb; 150 kb; 125 kb; 120 kb; 100 kb; 0.9 kb; 0.8 kb; 0.7 kb; 0.6 kb; 0.5 kb; 0.4 kb; 0.3 kb; 0.2 kb; or 0.1 kb of the original marker on the chromosome.

A probe may be an exact copy of a marker to be detected. A probe may also be a nucleic acid molecule comprising, or consisting of, a nucleotide sequence that is substantially identical to a cloned segment of chromosomal DNA comprising a marker to be detected (for example, as defined by SNP ID in Table 2 (maize)).

A probe may also be a nucleic acid molecule that is “specifically hybridizable” or “specifically complementary” to an exact copy of the marker to be detected (“DNA target”). “Specifically hybridizable” and “specifically complementary” are terms that indicate a sufficient degree of complementarity such that stable and specific binding occurs between the nucleic acid molecule and the DNA target. A nucleic acid molecule need not be 100% complementary to its target sequence to be specifically hybridizable. A nucleic acid molecule is specifically hybridizable when there is a sufficient degree of complementarity to avoid non-specific binding of the nucleic acid to non-target sequences under conditions where specific binding is desired, for example, under stringent hybridization conditions.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Calculations regarding hybridization conditions required for attaining particular degrees of stringency are known to those of ordinary skill in the art, and are discussed, for example, in Sambrook et al. (ed.) Molecular Cloning: A Laboratory Manual, 2^{nd} ed., vol. 1-3, Cold Spring Harbor Laboratory Press, Cold Spring Harbor, N.Y., 1989, chapters 9 and 11; and Hames and Higgins (eds.) Nucleic Acid Hybridization, IRL Press, Oxford, 1985. Further detailed instruction and guidance with regard to the hybridization of nucleic acids may be found, for example, in Tijssen, “Overview of principles of hybridization and the strategy of nucleic acid probe assays,” in Laboratory Techniques in Biochemistry and Molecular Biology-Hybridization with Nucleic Acid Probes, Part I, Chapter 2, Elsevier, NY, 1993; and Ausubel et al., Eds., Current Protocols in Molecular Biology, Chapter 2, Greene Publishing and Wiley-Interscience, NY, 1995.

As used herein, “stringent conditions” encompass conditions under which hybridization will only occur if there is less than 25% mismatch between the hybridization molecule and the DNA target. “Stringent conditions” include further particular levels of stringency. Thus, as used herein, “moderate stringency” conditions are those under which molecules with more than 25% sequence mismatch will not hybridize; conditions of “medium stringency” are those under which molecules with more than 15% mismatch will not hybridize; and conditions of “high stringency” are those under which sequences with more than 10% mismatch will not hybridize. Conditions of “very high stringency” are those under which sequences with more than 6% mismatch will not hybridize.

In particular embodiments, stringent conditions are hybridization at 65° C. in 6× saline-sodium citrate (SSC) buffer, 5×Denhardt's solution, 0.5% SDS, and 100 ng sheared salmon testes DNA, followed by 15-30 minute sequential washes at 65° C. in 2×SSC buffer and 0.5% SDS, followed by 1×SSC buffer and 0.5% SDS, and finally 0.2×SSC buffer and 0.5% SDS.

With respect to all probes discussed, supra, the probe may comprise additional nucleic acid sequences, for example, promoters; transcription signals; and/or vector sequences.

As used herein, linkage between genes or markers refers to the phenomenon in which genes or markers on a chromosome show a measurable probability of being passed on together to individuals in the next generation. The closer two genes or markers are to each other, the closer to (1) this probability becomes. Thus, the term “linked” may refer to one or more genes or markers that are passed together with a second gene or marker with a probability greater than 0.5 (which is expected from independent assortment where markers/genes are located on different chromosomes). Because the proximity of two genes or markers on a chromosome is directly related to the probability that the genes or markers will be passed together to individuals in the next generation, the term “linked” may also refer herein to one or more genes or markers that are located within about 2.0 Mb of one another on the same chromosome. Thus, two “linked” genes or markers may be separated by about 2.1 Mb; 2.00 Mb; about 1.95 Mb; about 1.90 Mb; about 1.85 Mb; about 1.80 Mb; about 1.75 Mb; about 1.70 Mb; about 1.65 Mb; about 1.60 Mb; about 1.55 Mb; about 1.50 Mb; about 1.45 Mb; about 1.40 Mb; about 1.35 Mb; about 1.30 Mb; about 1.25 Mb; about 1.20 Mb; about 1.15 Mb; about 1.10 Mb; about 1.05 Mb; about 1.00 Mb; about 0.95 Mb; about 0.90 Mb; about 0.85 Mb; about 0.80 Mb; about 0.75 Mb; about 0.70 Mb; about 0.65 Mb; about 0.60 Mb; about 0.55 Mb; about 0.50 Mb; about 0.45 Mb; about 0.40 Mb; about 0.35 Mb; about 0.30 Mb; about 0.25 Mb; about 0.20 Mb; about 0.15 Mb; about 0.10 Mb; about 0.05 Mb; about 0.025 Mb; about 0.012 Mb; and about 0.01 Mb. As used herein, the term “tightly linked” may refer to one or more genes or markers that are located within about 0.5 Mb of one another on the same maize chromosome. As used herein, the term “extremely tightly linked” may refer to one or more genes or markers that are located within about 100 kb of one another on the same maize chromosome.

As used herein, linkage between markers and traits or phenotypes of interest may refer to one or more markers that are each passed together with a trait or phenotype with a probability greater than expected from random chance (0.5). While a marker may be comprised in some examples within a gene that determines a particular trait or phenotype, it will be understood that most often a marker may be separated by a short distance (e.g., less than about 2 Mb) from such a gene on the same chromosome. Moreover, it will be understood that most traits or phenotypes are polygenic, and thus a marker that is linked to a trait or phenotype may in some examples reside within, or be linked to, a QTL underlying a polygenic trait.

Linked, tightly linked, and extremely-tightly linked genetic markers may be useful in marker-assisted breeding programs, for example and without limitation, to introgress a trait or phenotype of interest into a plant variety; and to generate new plant varieties comprising a trait or phenotype of interest.

Marker-assisted breeding: As used herein, the term “marker-assisted breeding” may refer to an approach to breeding directly for one or more trait(s) (e.g., a polygenic trait). In current practice, plant breeders attempt to identify easily detectable traits, such as flower color, seed coat appearance, or isozyme variants that are linked to an agronomically desired trait. The plant breeders then follow the agronomic trait in the segregating, breeding populations by following the segregation of the easily detectable trait. However, there are very few of these linkage relationships between traits of interest and easily detectable traits available for use in plant breeding. In some embodiments of the invention, marker-assisted breeding comprises identifying one or more genetic markers (e.g., SNP markers) that are linked to a trait of interest, and following the trait of interest in a segregating, breeding population by following the segregation of the one or more genetic markers. In some examples, the segregation of the one or more genetic markers may be determined utilizing a probe for the one or more genetic markers by assaying a genetic sample from a progeny plant for the presence of the one or more genetic markers.

Marker-assisted breeding provides a time- and cost-efficient process for improvement of plant varieties. Several examples of the application of marker-assisted breeding involve the use of isozyme markers. See, e.g., Tanksley and Orton, eds. (1983) Isozymes in Plant Breeding and Genetics, Amsterdam: Elsevier. One example is an isozyme marker associated with a gene for resistance to a nematode pest in tomato. The resistance, controlled by a gene designated Mi, is located on chromosome 6 of tomato and is very tightly linked to Aps1, an acid phosphatase isozyme. Use of the Aps1 isozyme marker to indirectly select for the Mi gene provided the advantages that segregation in a population can be determined unequivocally with standard electrophoretic techniques; the isozyme marker can be scored in seedling tissue, obviating the need to maintain plants to maturity; and co-dominance of the isozyme marker alleles allows discrimination between homozygotes and heterozygotes. See Rick (1983) in Tanksley and Orton, supra.

Optimized: As used herein in the context of a panel of genetic markers, the term “optimized” refers to a panel of markers that performs better (e.g., provides greater GA or LD coverage) than a reference panel comprising the same number of non-identical markers in predicting the presence or absence of a trait of interest. Thus, in some examples, an “optimized” marker panel is a subset of a large number of genetic markers in a plant species that performs better in predicting the presence or absence of a trait of interest or donor DNA than a different subset of the same size consisting of markers from the large number of genetic markers. In some examples, an “optimized” marker panel is a subset of a set of genetic markers in a plant species that retains more of the predictive value (for the presence or absence of a trait of interest) of the entire set of genetic markers than a different subset of the same size consisting of markers from the entire set of genetic markers.

The term “optimized” may refer to a subset that provides the best performance over all other subsets, but this is not necessarily the case. An optimized marker set may be further optimized to provide even better performance, for example, by performing further iterations of an ACO system, or by performing iterations of an ACO system in the presence of additional segregation data.

Sequence identity: The term “sequence identity” or “identity,” as used herein in the context of two nucleic acid sequences, may refer to the nucleobases in the two sequences that are the same when aligned for maximum correspondence over a specified comparison window.

As used herein, the term “percentage of sequence identity” may refer to the value determined by comparing two optimally aligned nucleic acid sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleobase occurs in both sequences to yield the number of matched positions, dividing the number of matched positions by the total number of positions in the comparison window, and multiplying the result by 100 to yield the percentage of sequence identity.

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using default parameters. Nucleic acid sequences with even greater similarity to the reference sequences will show increasing percentage identity when assessed by this method.

As used herein, the term “substantially identical” may refer to nucleotide sequences that are more than 85% identical. For example, a substantially identical nucleotide sequence may be at least 85.5%; at least 86%; at least 87%; at least 88%; at least 89%; at least 90%; at least 91%; at least 92%; at least 93%; at least 94%; at least 95%; at least 96%; at least 97%; at least 98%; at least 99%; or at least 99.5% identical to the reference sequence.

Single-nucleotide polymorphism (SNP): As used herein, the term “single-nucleotide polymorphism” may refer to a DNA sequence variation occurring when a single nucleotide in the genome (or other shared sequence) differs between members of a species or paired chromosomes in an individual.

Within a population, SNPs can be assigned a minor allele frequency of the lowest allele frequency at a locus that is observed in a particular population. This is simply the lesser of the two allele frequencies for single-nucleotide polymorphisms. There are variations between plant populations, so an SNP allele that is common in one population may be rarer in a different population.

Single nucleotide polymorphisms may fall within coding sequences of genes, non-coding regions of genes, or in the intergenic regions between genes. SNPs within a coding sequence will not necessarily change the amino acid sequence of the protein that is produced, due to degeneracy of the genetic code. An SNP in which both forms lead to the same polypeptide sequence is termed “synonymous” (sometimes called a silent mutation). If a different polypeptide sequence is produced, they are termed “non-synonymous.” A non-synonymous change may either be missense or nonsense, where a missense change results in a different amino acid, and a nonsense change results in a premature stop codon. SNPs that are not in protein-coding regions may still have consequences for gene splicing, transcription factor binding, or the sequence of non-coding RNA. SNPs are usually biallelic and thus easily assayed in plants and animals. Sachidanandam (2001) Nature 409:928-33.

Stigmergy: As used herein, the term “stigmergy” or “stigmergic communication” refers to indirect communication between agents mediated by physical modifications of environmental state variables, the values of which are only locally accessible by the communicating agents (i.e., ants).

Trait or phenotype: The terms “trait” and “phenotype” are used interchangeably herein. For the purposes of the present disclosure, traits of particular interest include agronomically important traits, as may be expressed, for example, in a crop plant.

### IV. Markers for Use in Plant Breeding

Embodiments of the invention include genetic markers in a plant that may be linked to a trait of interest. Some embodiments include a set of markers in the genome of a plant from which may be identified, through the implementation of an ACO system, a subset of markers that may be used to predict the presence or absence of a trait of interest in a plant from which a genetic sample has been provided. Sets of genetic markers, and optimized subsets identified therefrom, may comprise one or more markers that are individually linked to the trait of interest.

Some markers that may be used in particular embodiments are known in the art. For example, genetic markers have been made available in many plant species through genome sequencing, genotyping, and QTL mapping studies. Additional markers that may be used in particular embodiments may be identified by any technique known to those of skill in the art, including for example and without limitation: molecular techniques such as RAPD, identification of RFLPs, AFLP-PCR, DAF, identification of SCARS, and/or identification of microsatellites; and direct comparison of aligned genomic nucleic acid sequences from several populations.

In some examples, a set of markers comprises SNP markers. The genotyping of a plant for one or more SNP markers is easily carried out, for example, by using one of many PCR-based analysis techniques. In particular examples, the genotyping of a plant over a set of SNP markers may be carried out by utilizing the OpenArray® SNP genotyping system (Applied BioSystems). The OpenArray® system uses “chips” comprising a panel of SNPs to determine the genotype of an organism from which a genetic sample has been provided, by assaying for specific hybridization of nucleic acids within the genetic sample to the panel of SNPs.

### V. Ant Colony Optimization

ACO is an optimization method that was designed by reference to the natural process of pheromone use in ants to identify the shortest path from a spatial point of interest to the nest. Dorigo and Gambardella (1997) BioSystems 43:73-81; Dorigo et al. (1999) Artificial Life 5:137-72. In nature, ants each deposit a certain amount of pheromone while walking, and each ant probabilistically prefers to follow a direction rich in pheromone. If an obstacle appears along a path between a spatial point of interest (e.g., a food source) and the nest, those ants approaching the obstacle must choose between turning right or left to avoid the obstacle. In the absence of a pheromone cue providing direction one way or the other, half the ants will choose to turn right, and the other half will choose to turn left. Those ants which choose, by chance, the shorter path around the obstacle will reconstitute an uninterrupted pheromone trail more rapidly than those who choose a longer path. This behavior establishes an autocatalytic process, whereby the shorter path receives a greater amount of pheromone per time unit, and a larger number of ants consequently chooses the shorter path. If this process is allowed to reach its natural conclusion, all the ants will rapidly choose the shorter path.

The shortest path around the obstacle may be thought of as an emergent property of the interaction between the shape of the obstacle and the distributed behavior of the ants. Although all the ants move at approximately the same speed and deposit a pheromone trail at approximately the same rate, it takes longer to contour obstacles on their longer side than on their shorter side. Consequently, the pheromone trail accumulates more rapidly on the shorter side. The ants' preference for a trail with higher pheromone levels makes this accumulation still quicker on the shorter path. According to the foregoing, while each ant may find a solution (i.e., a path between the two points), only the activity of a collection of m ants leads to optimization.

In an exemplary ACO system, m ants are placed on randomly selected spatial points comprised within an appropriate representation of the problem to be solved. At each time step, the ants move to new points and may modify the pheromone trail on the edges (i.e., paths between points) used, in a process referred to as “local trail updating.” When all the ants have completed a movement, the ant that made the shortest movement may modify the edge belonging to its movement (“global trail updating”) by adding an amount of pheromone trail that is inversely proportional to the movement length. In some embodiments, ants may be able to determine the distance between points, and/or have a working memory (Mₖ) used to memorize points already visited (the working memory may be emptied at the beginning of each new movement, and may be updated after each time step).

Ants are tasked to find a shortest path joining an initial problem situation to a destination situation. Ants must move step-by-step through adjacent problem states. Ants build solutions through applying a probabilistic decision policy to move through adjacent states, in most embodiments making use only of local information without prediction of future states. Thus, the decision policy may be completely local in space and time. The decision policy is a function of the a priori information represented by the problem specifications, and local modifications in the problem environment (pheromone trails) induced by ants in the past. Once an individual ant has built a problem solution and deposited pheromone information, the ant may be deleted from the ACO system. Although the complexity of each ant is such that it can build a feasible solution (as a real ant can somehow find a path between the nest and the food), high-quality solutions are the result of the cooperation among the individuals of the whole colony.

In some embodiments, features of an ACO system of the invention may include, for example and without limitation, a plurality of cooperating individual agents (a colony of ants); an artificial “pheromone” trail (i.e., numeric information that takes into account the depositing ant's current history or performance and can be read/written by any ant accessing the state) that modifies the local state of a problem for local stigmergetic communication; a sequence of local moves to find shortest paths; and a stochastic decision policy using local information without prediction of future states. In some embodiments, features of an ACO system of the invention may also include, for example and without limitation, a discrete problem environment comprising discrete adjacent states, wherein the ants' movements consist of transitions between discrete adjacent states; internal states in each ant comprising memory of the ant's past actions; and deposition of pheromone in an amount that is a function of the quality of the solution found.

In some embodiments, stigmergetic communication provided by local pheromone trails may be the only communication channel between the ants. However in some embodiments, some prediction of future states may be employed. Michel and Middendorf (1998) “An island model based Ant System with lookahead for the shortest supersequence problem.” In Proceedings of PPSN-V, Fifth International Conference on Parallel Problem Solving from Nature, Eiben et al. (Eds.), Springer-Verlag, Berlin.

In some embodiments, a stochastic component of the ants' decision policy and/or an “evaporation mechanism” may prevent ants from being constrained by past decisions to rapidly migrate toward a same previously-visited part of the search space. An evaporation mechanism modifies the information in local pheromone trails over time, so that the ant colony may forget or partially forget its past history. A stochastic component of the ants' decision policy determines the balance between the exploration of new points in the state space and the exploitation of accumulated knowledge according to the level of stochasticity in the policy, and the strength of the updates in the local pheromone trails. A particular level of stochasticity and/or a strength of pheromone trail updates, as well as the strength of an evaporation mechanism, may be determined in embodiments according to the discretion of the practitioner.

In some examples, the ants' timing in deposition of pheromone is problem dependent. For example, ants may update pheromone trails only after having generated a solution. Also in some examples, an ACO system may be enriched with capabilities including, for example and without limitation, local optimization (see, e.g., Dorigo and Gambardella (1997) IEEE Transactions on Evolutionary Computation 1(1):53-66); backtracking/recovery procedures (see Di Caro and Dorigo (1998) J. Art. Intel. Res. (JAIR) 9:317-65); and an extra-ant component that may observe the ants' behavior to collect useful global information and deposit additional pheromone information that biases the ants' search processes from a non-local perspective (Dorigo et al. (1999), supra). These and other modifications may improve, for example, the efficiency and/or performance of the ACO system. In an ACO system, ant generation and activity, pheromone evaporation, and extra-ant activity may be synchronized during the performance of the system. In some examples, a sequential scheduling of system activities is used.

In embodiments of the invention, an optimized panel of genetic markers from a plant is identified through the implementation of an ACO system. In some embodiments, the spatial points in the problem space may correspond to discrete subsets of markers from a larger discrete marker set. In some embodiments, the stigmergetic communication between the ants may be represented by a PDF that is updated by pheromone levels that are determined by the genome (GA) and linkage drag (LD) coverage provided by the selected markers for a trait of interest. Performance of an ACO system according to such embodiments through multiple time steps may identify a panel of genetic markers that is optimized for the identification and/or introgression of the trait of interest. In particular examples, the larger discrete marker set may comprise at least about 500 markers; at least about 600 markers; at least about 700 markers; at least about 800 markers; at least about 900 markers; at least about 1000 markers; at least about 1100 markers; at least about 1200 markers; at least about 1300 markers; at least about 1400 markers; at least about 1500 markers; at least about 1600 markers; at least about 1700 markers; at least about 1800 markers; at least about 1900 markers; at least about 2000 markers; or more.

To test the effectiveness of an ACO system in identifying an optimized panel of genetic markers from a plant, the ACO may be applied to multiple populations of the plant that are targeted for introgression of the trait of interest. In particular examples, the ACO may be applied to more than about 100 populations; less than about 100 populations; less than about 90 populations; less than about 80 populations; less than about 75 populations; less than about 70 populations; less than about 60 populations; less than about 50 populations; or fewer. The effectiveness of the ACO system may be evaluated by comparing the GA and LD coverage obtained using an identified, optimized marker subset, to the coverage obtained when using all of the markers in the larger set from which the optimized marker subset was identified. Thus evaluated, the effectiveness of the ACO system may be compared to alternative panel selection methods. Such an optimized marker subset may provide better GA and/or LD coverage than alternative methods across a number of trait introgression projects.

General information regarding ACO systems and their implementation may be found in, for example, Dorigo et al. (1999), supra.

### VI. Use of Optimized Marker Panels

Some embodiments include methods of identifying plants likely to comprise a trait of interest using optimized molecular marker panels that have been identified by a process utilizing an ACO system. In particular embodiments, nucleic acid molecules (e.g., genomic DNA or mRNA) may be extracted from a plant. The extracted nucleic acid molecules may then be contacted with one or more probes that are specifically hybridizable to markers in an optimized marker panel. Specific hybridization of the one or more probes to the extracted nucleic acid molecules is indicative of the presence of the trait of interest in the plant. Such methods may result in a cost savings for plant developers, because use of such methods may eliminate the need to phenotype individual plants generated during development (for example, by crossing a plant variety having a trait of interest with a plant variety lacking the trait of interest).

In some embodiments, optimized marker panels that have been identified by a process utilizing an ACO system to be predictive of a trait of interest may be used to transfer segment(s) of DNA that contain one or more genes or QTLs that determine or contribute to the trait of interest (i.e., trait introgression). In particular embodiments, a method for using such an optimized marker panel may comprise, for example and without limitation, providing a first parent plant comprising markers in the optimized marker panel; providing a second parent plant; analyzing the genomic DNA of the first and second parent plants with probes that are specifically hybridizable to markers in the optimized marker panel; crossing the two parental plant genotypes to obtain a progeny population, and analyzing those progeny for the presence of the markers in the optimized marker panel; backcrossing the progeny that contain the markers in the optimized marker panel to the second parental genotype to produce a first backcross population, and then continuing with a backcrossing program until a final progeny is obtained that comprises any desired trait(s) exhibited by the second parent genotype and the markers in the optimized marker panel, thereby transferring the segment(s) of DNA that contain one or more genes or QTLs that determine or contribute to the trait of interest. In particular embodiments, the progeny of the first cross, or any subsequent backcross, may be crossed to a third plant that is of a different line or genotype than either the first or second plant. A final progeny plant that comprises any desired trait(s) exhibited by the second parent genotype and the markers in the optimized marker panel may be likely to comprise the trait of interest.

In some examples, individual progeny obtained in each crossing and backcrossing step are selected by marker panel analysis at each generation. In some examples, analysis of the genomic DNA of the two parent plants with probes that are specifically hybridizable to the markers in the optimized marker panel reveals that one of the parent plants comprises fewer of the markers to which the probes specifically hybridize, or none of the markers to which the probes specifically hybridize. In some examples, the first parent plant may comprise the trait of interest, or may lack the trait of interest but comprise a genotype that is predictive of the trait of interest.

According to the foregoing, progeny plants may be subjected in some examples to a genotype and/or zygosity determination. Once progeny plants have been genotyped, and/or their zygosity has been determined, the skilled artisan may select those progeny plants that have a desired genetic composition (e.g., progeny plants that comprise markers of an optimized marker panel). Such selected progeny plants may be used in further crosses, selfing, or cultivation. Methods of trait introgression that employ optimized marker panels identified by a process utilizing an ACO system to be predictive of the trait may reduce or eliminate the cultivation and/or reproduction of plants that do not have the desired genetic composition, and thereby provide desirable reliability and predictability (through expected Mendelian patterns of inheritance) in selective plant breeding or development programs.

The following examples are provided to illustrate certain particular features and/or embodiments. The examples should not be construed to limit the disclosure to the particular features or embodiments exemplified.

### EXAMPLES

### Example 1

### Materials and Methods

Data. The dataset consisted of genotype information on 72 recurrent inbred maize lines targeted for trait introgression, and five inbred maize lines serving as donors. Each line was genotyped for 1371 markers available for use with the OpenArray® SNP genotyping system. For each recurrent and donor parent combination, markers were scored as either informative or uninformative based on polymorphisms between the two parents.

SNP panel selection. Three sampling methods were used to select subsets of markers (Sₖ): random sampling (RS), sampling based on prior information (PS) (prior information is computed as the polymorphic rate of a SNP), and the ACA. RS sampled marker subsets at random, and the probability of a marker being selected by PS was based on the proportion of times a marker was informative for the 72 recurrent and donor parent combinations. The ACO sampling method used an ant colony sampling method. Each set of markers was evaluated based on the GA and LD coverage, calculated as:

$$\begin{matrix}{coverage}_{GA} = \frac{{\sum }_{i = 1}^{nm} M W G {A}_{i}}{genome length} \\ and & ( 1 ) \\ {coverage}_{LD} = \frac{{\sum }_{i = 1}^{nmi} M W L {D}_{i}}{{\sum }_{j = 1}^{ni} length insert {chromosome}_{j}} & ( 2 )\end{matrix}$$

where nm is the number of informative markers in Sₖ; nmi is the number of informative markers flanking an insert site; ni is the number of chromosomes with trait inserts; and MWGAᵢ and MWLDᵢ are marker weights for GA and LD, respectively, subject to the following restrictions:

$$M W G {A}_{i} = \{ \begin{matrix}20 cM & if weight of {marker}_{i} > 20 cM \\ marker {weight}_{i} & otherwise\end{matrix} \\ For M W L {D}_{i} , \\ if {marker}_{i} was further than 30 cM from the insert site , \\ M W L {D}_{i} = \{ \begin{matrix}10 cM & if weight of {marker}_{i} > 10 cM \\ marker {weight}_{i} & otherwise\end{matrix} \\ otherwise \\ M W L {D}_{i} = \{ \begin{matrix}5 cM & if weight of {marker}_{i} > 5 cM \\ marker {weight}_{i} & otherwise .\end{matrix}$$

Marker weights were calculated as the sum of half the distance (in cM) between the marker of interest and the nearest informative upstream and downstream markers in Sₖ.

Ant Colony Optimization. Artificial ants were defined as parallel units that communicate through a probability density function (PDF) that is updated by weights or “pheromone levels,” which in this case are determined by the GA and LD coverage provided by the selected markers. See Dorigo et al. (1999), supra; see also Ressom et al. (2007), supra; see also Robbins et al. (2007) Math. Med. Biol. 24:413-26. The probability of sampling marker m at time t was defined as:

$${P}_{m} ( t ) = \frac{{( {τ}_{m} ( t ) )}^{α} {η}_{m}^{β}}{{\sum }_{m = 1}^{nf} {( {τ}_{m} ( t ) )}^{α} {η}_{m}^{β}} \qquad ( 3 )$$

where τₘ (t) is the amount of pheromone for marker m (out of a total of nf markers) at time t, ηₘ is the prior information used by PS for marker m; α and β are parameters determining the weight given to pheromone deposited by ants and a priori information on the features, respectively. For this study, α and β were both set to 1.

The ACA was initialized with all markers having an equal baseline level of pheromone used to compute used to compute Pₘ(0) for all markers. Using the PDF as defined in Eq. 3, each of j ants will select a subset (Sₖ) of n markers from the sample space S containing all 1371 markers. The pheromone level of each marker m in Sₖ was then updated according to the performance of Sₖ as:

τₘ(t+1)=(1−ρ)*τₘ(t)+Δτₘ(t) (4)

where ρ is a constant between 0 and 1 that represents the rate at which the pheromone trail evaporates; Δτₘ(t) is the change in pheromone level for marker m based on the performance of Sₖ, and it is set to 0 if feature m∉Sₖ. This process is repeated for all Sₖ.

The procedure employed can be summarized by the following steps: First, each ant selects a predetermined number of markers; then, using the selected markers, performance is calculated as:

performance=0.5*coverage_{GA}+0.5*coverage_{LD} (5)

and third, the change in pheromone is calculated as:

Δτₘ(t)=performance^{(1-performance)} (6)

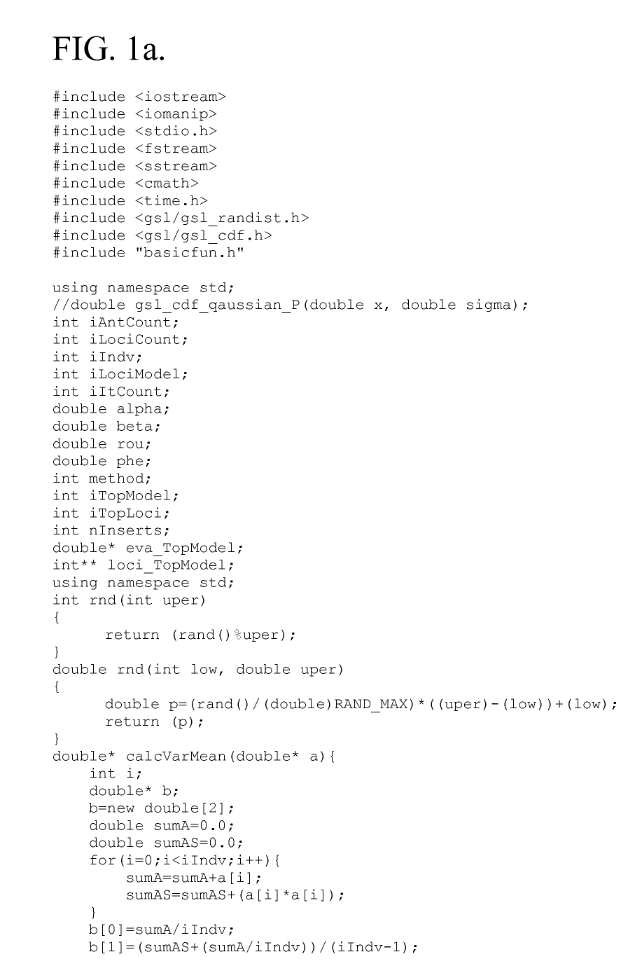

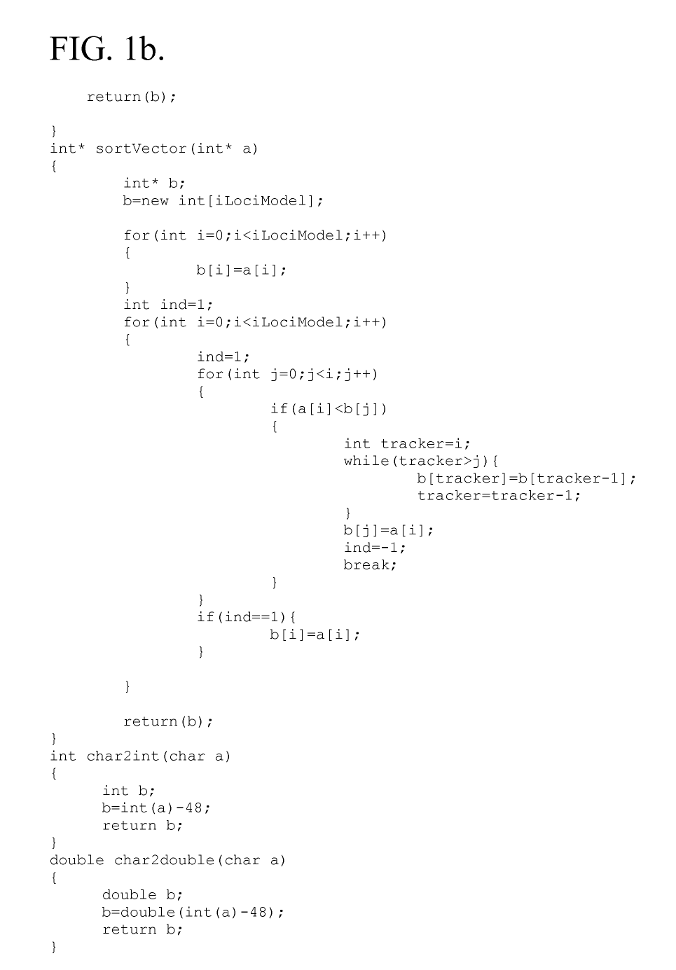

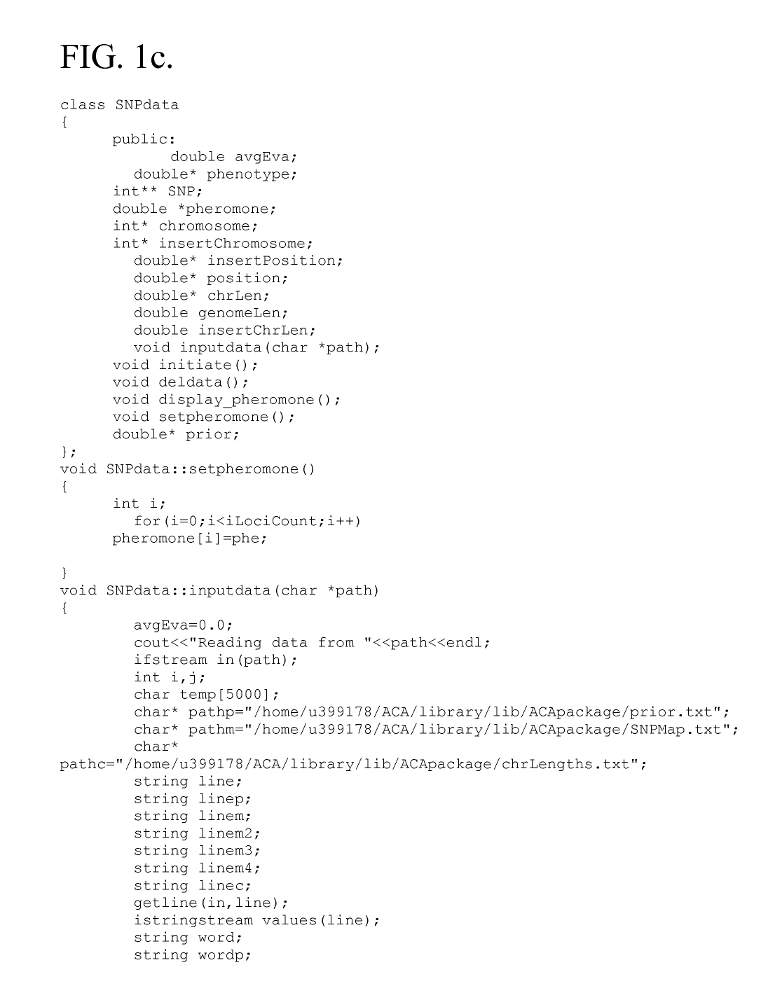

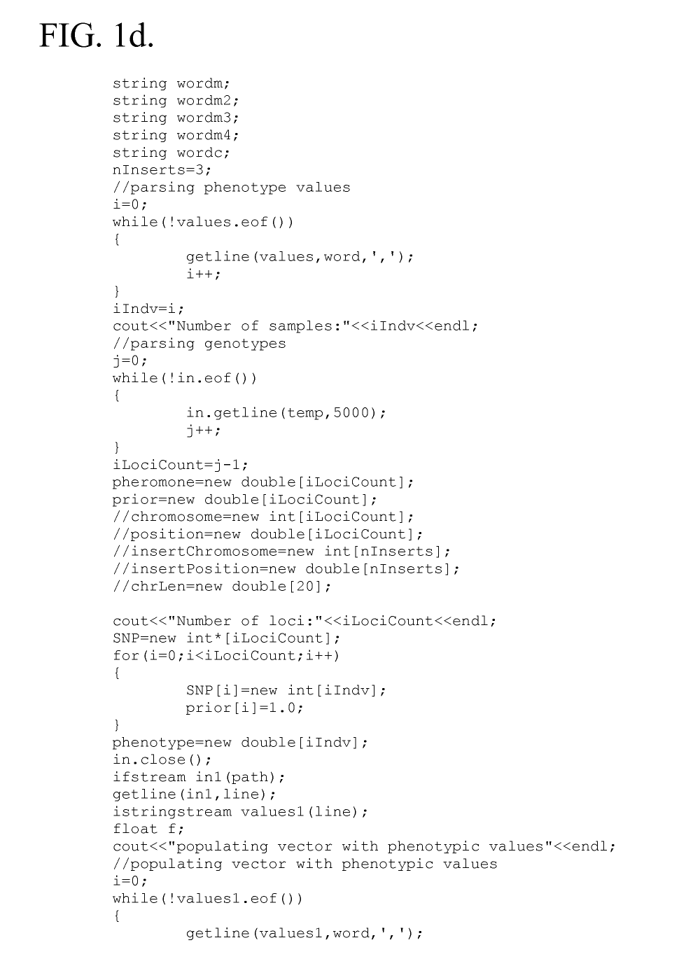

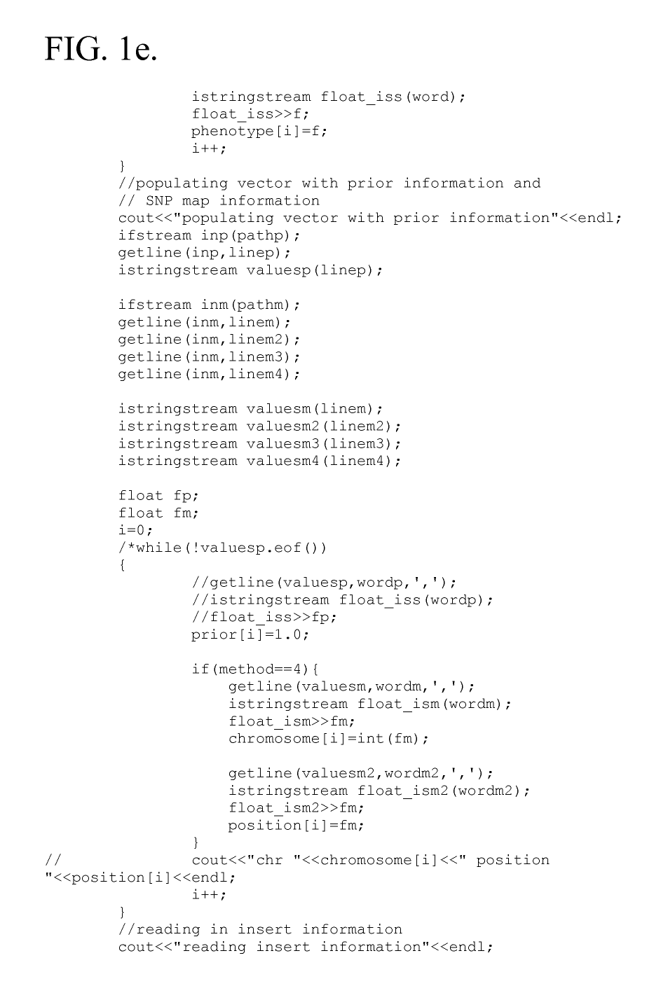

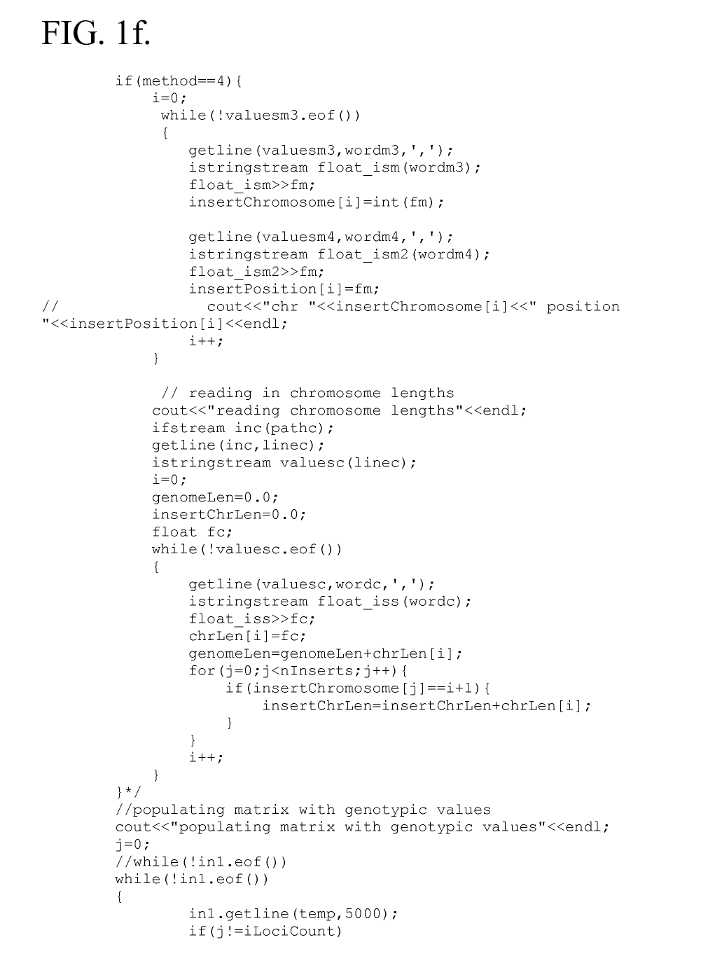

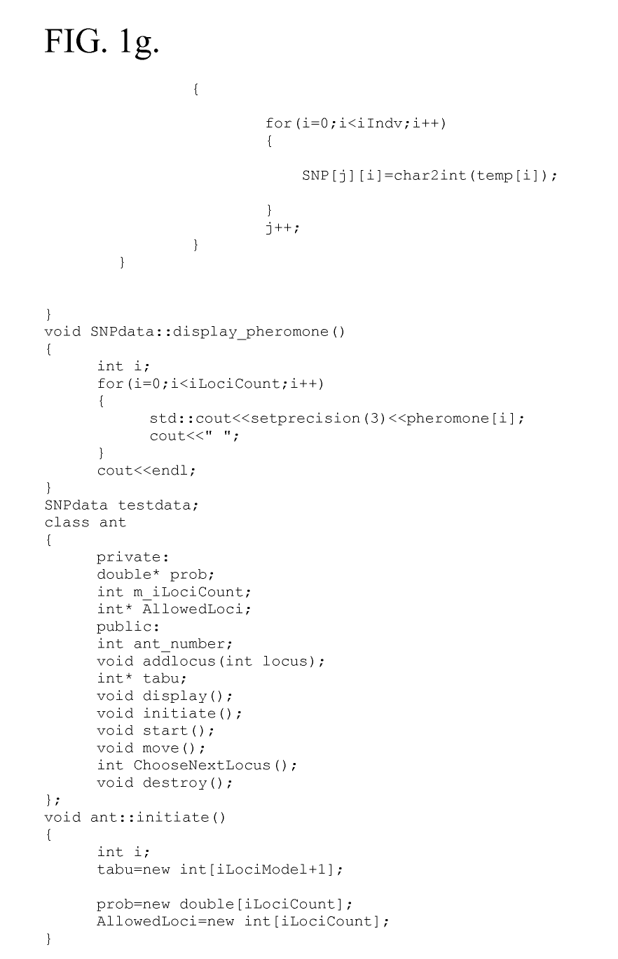

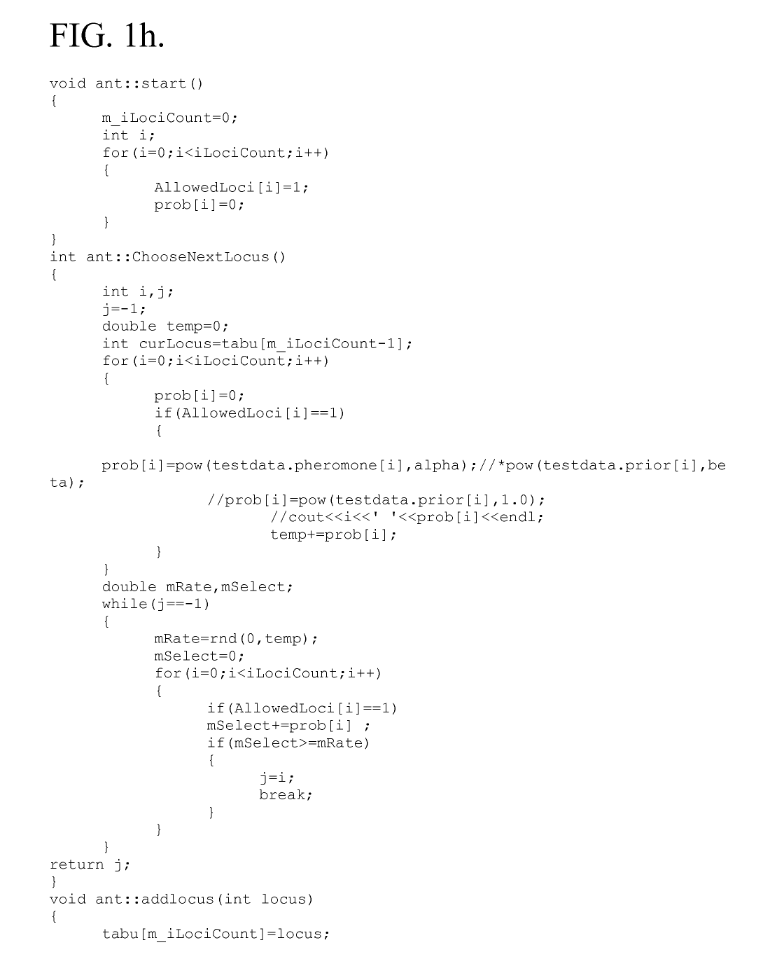

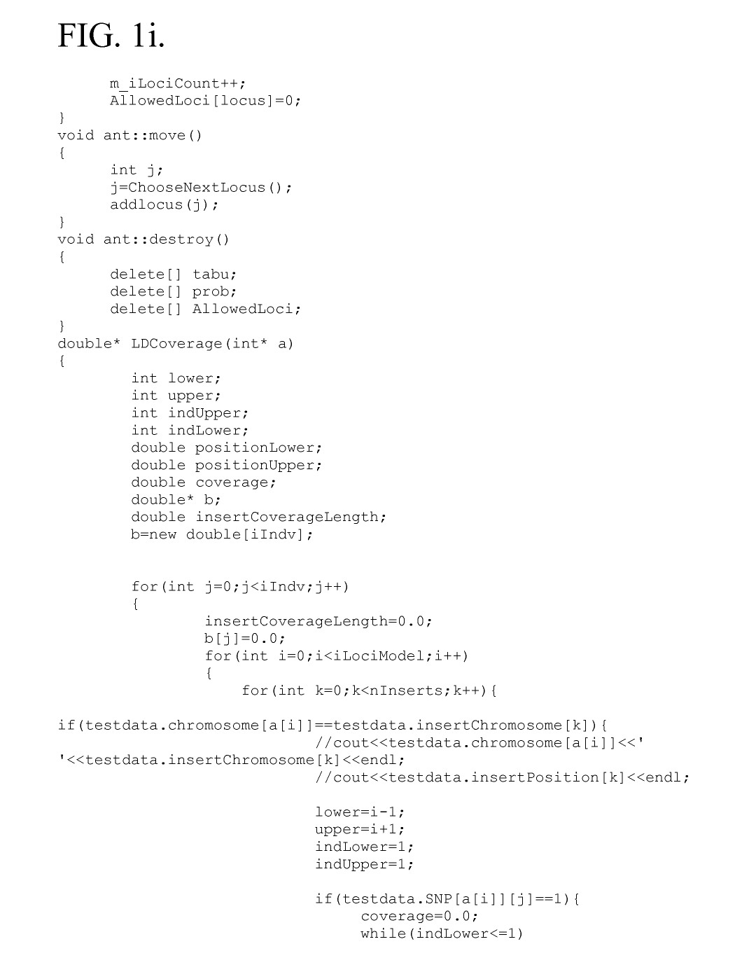

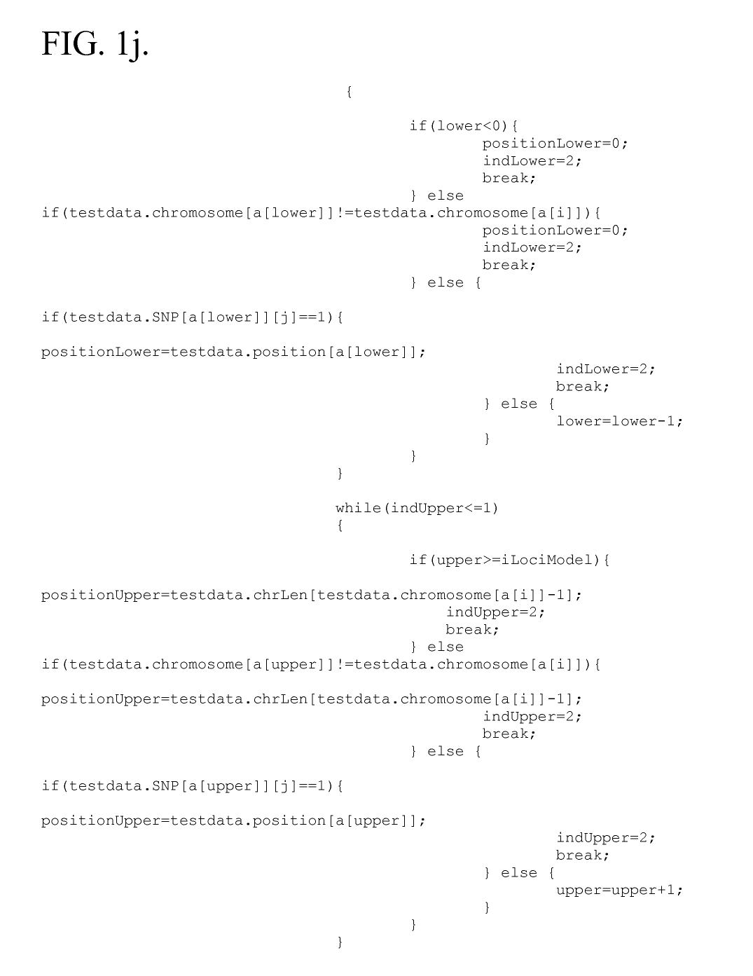

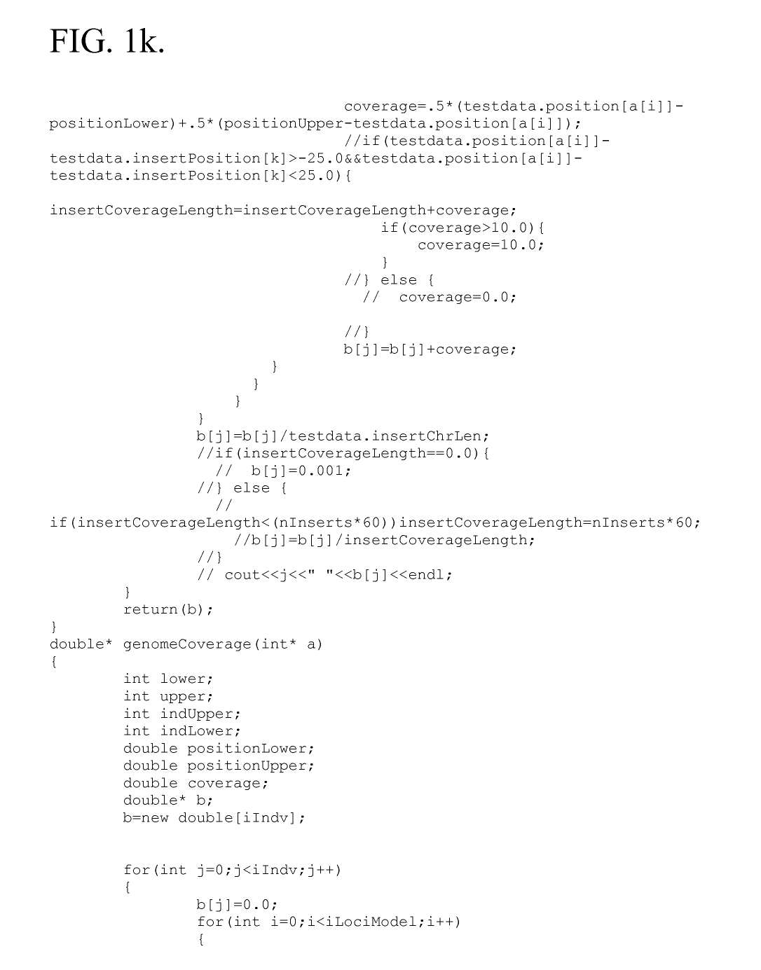

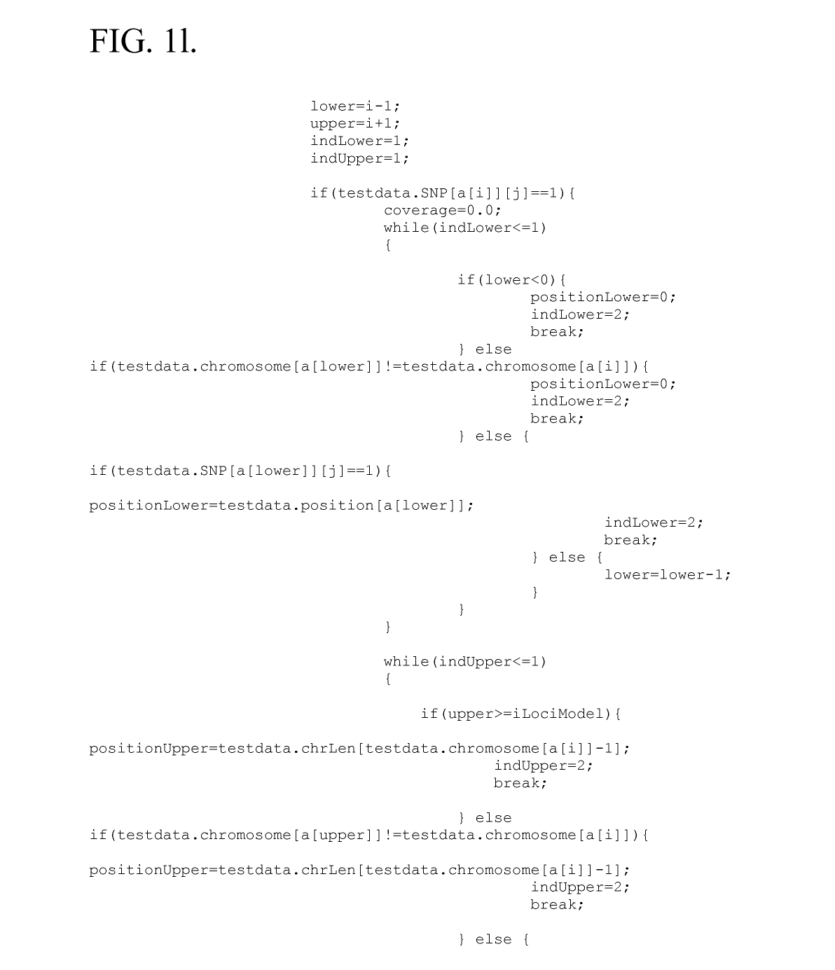

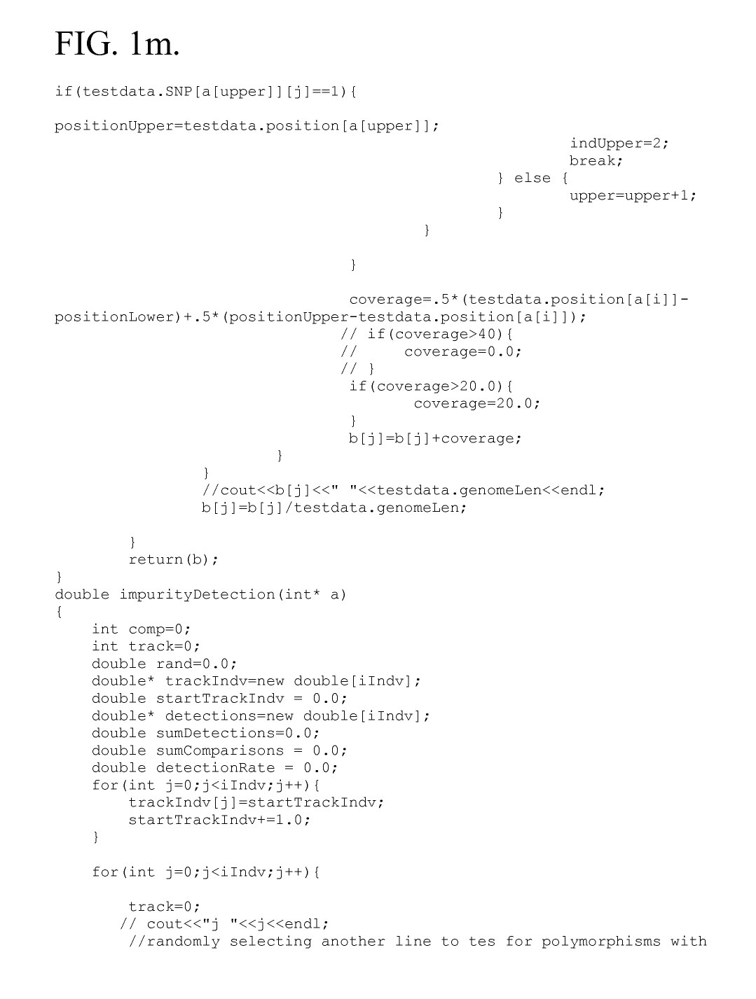

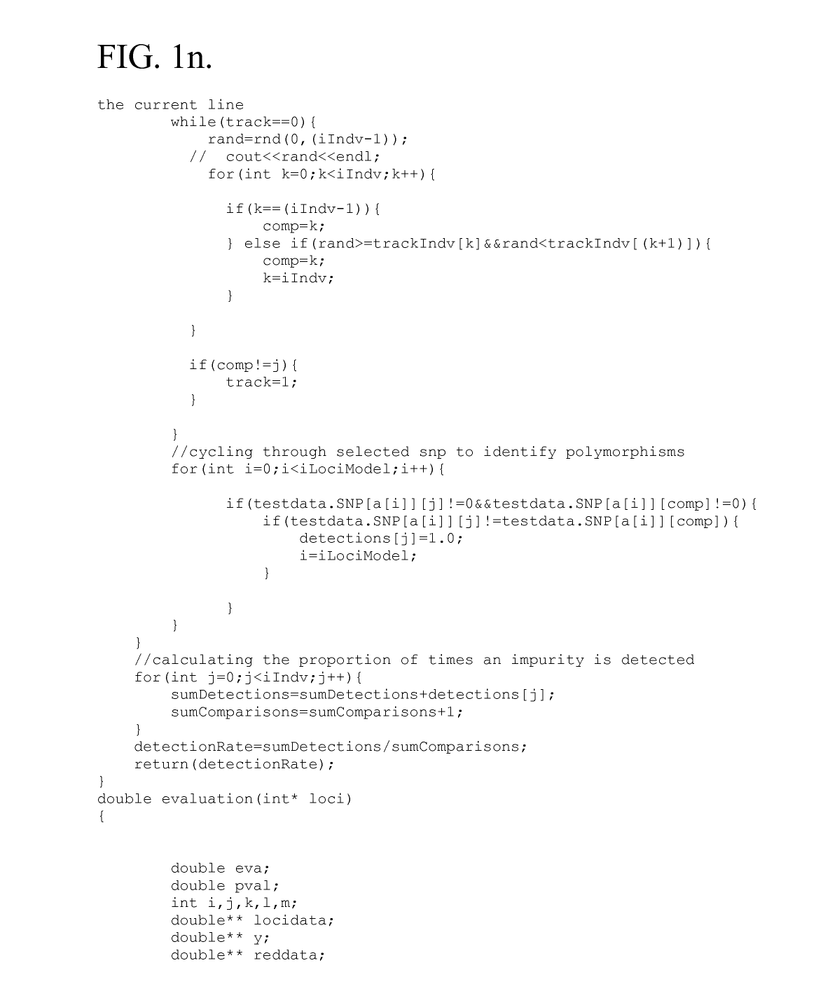

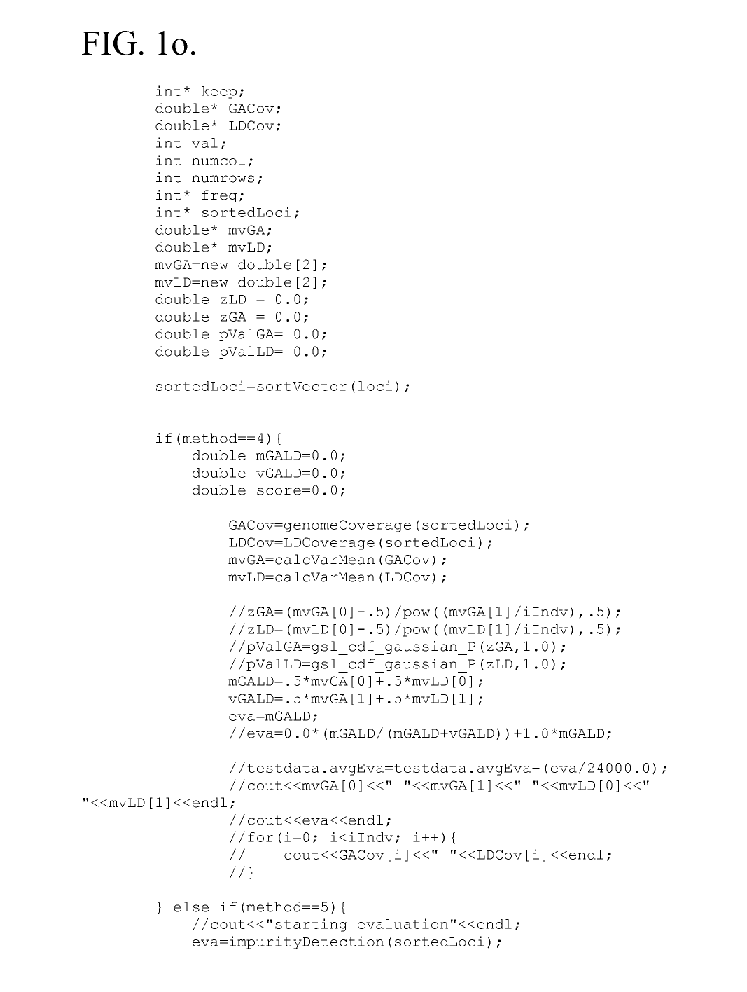

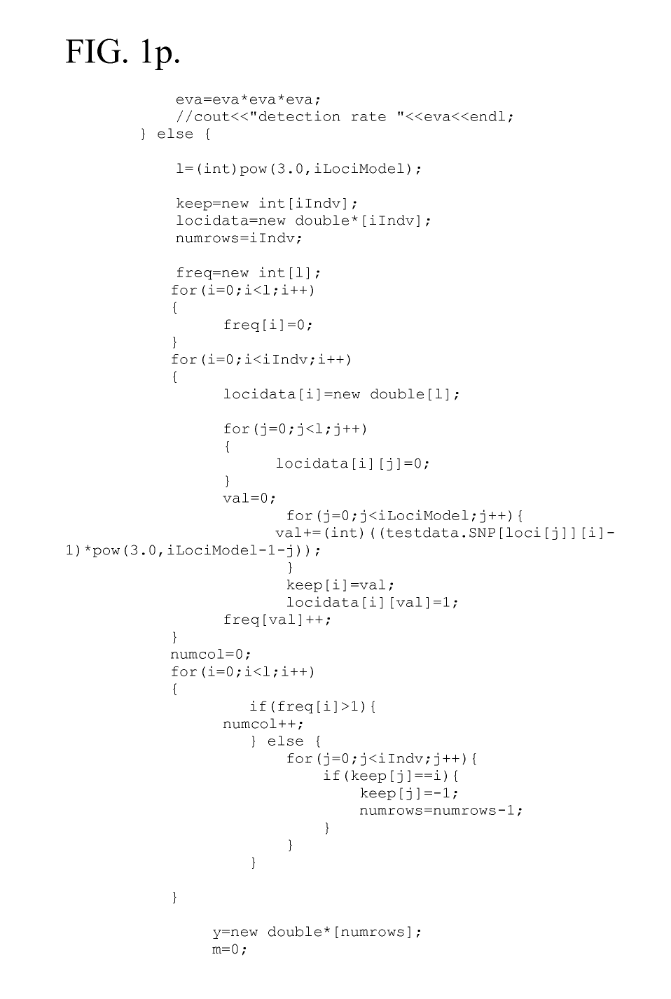

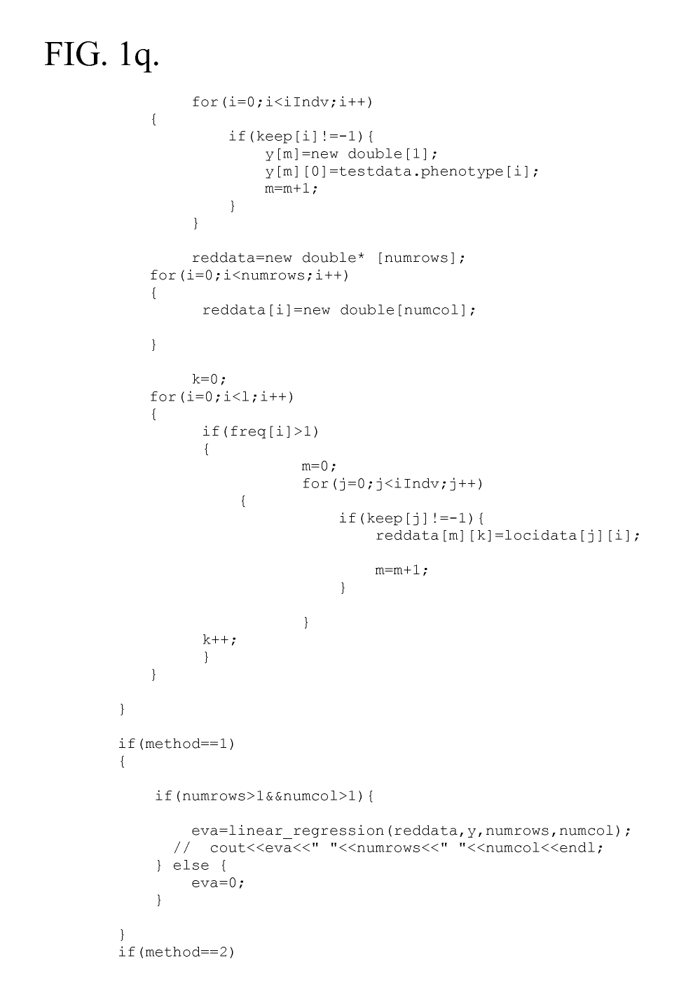

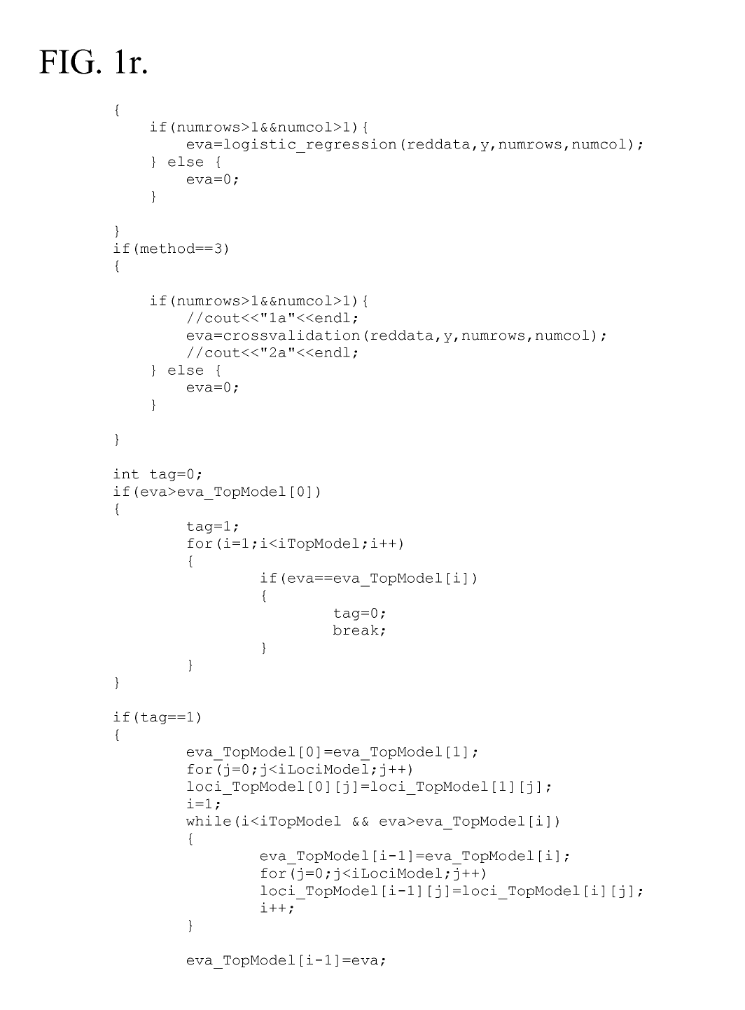

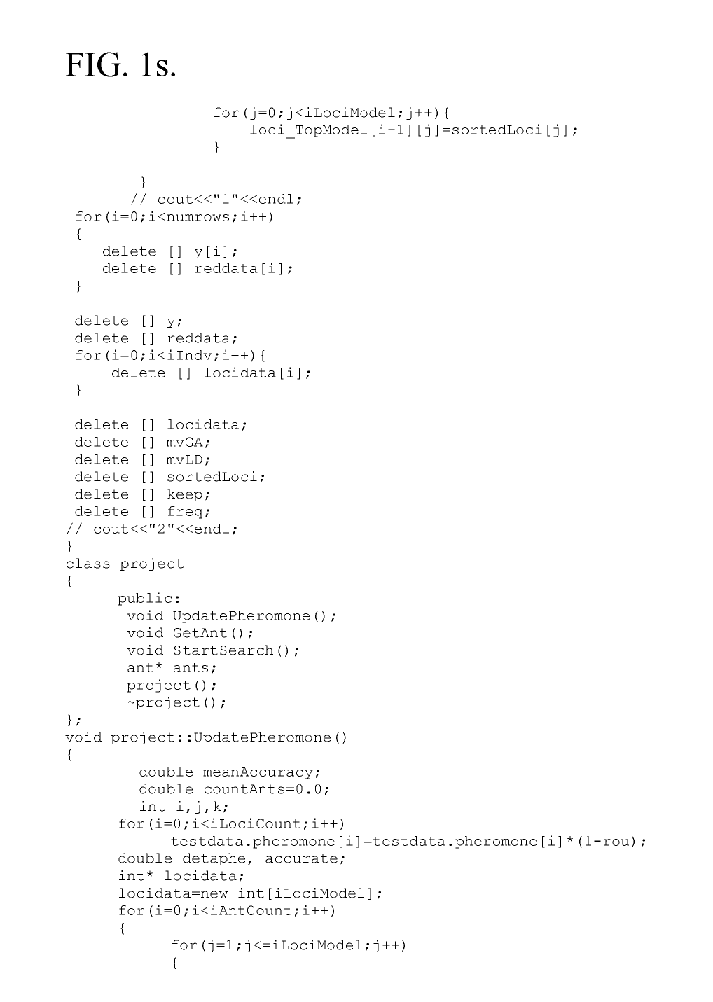

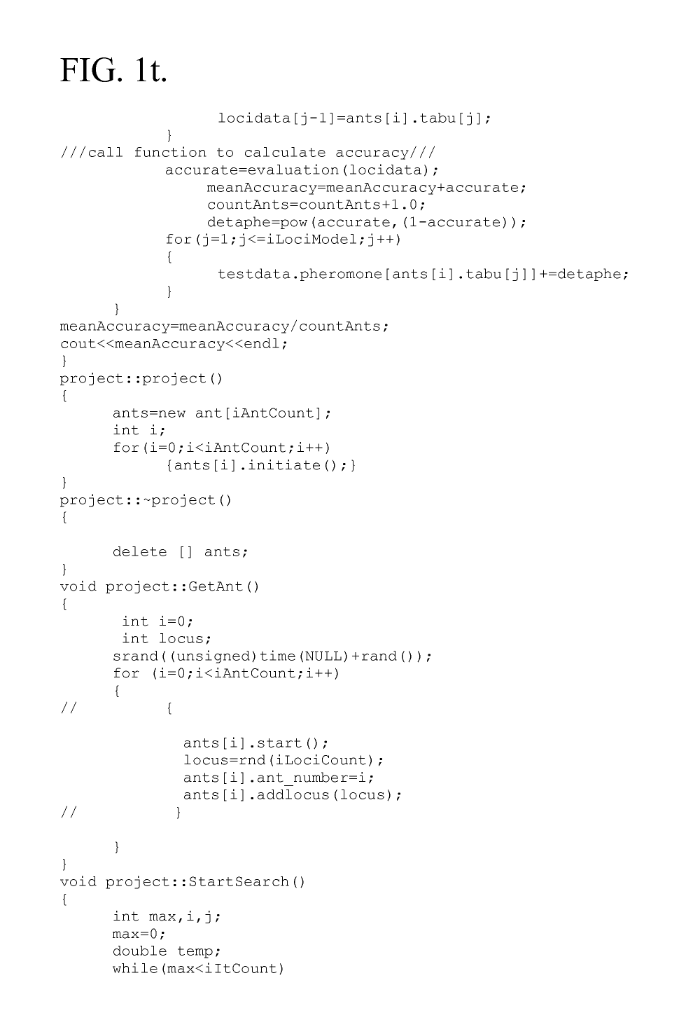

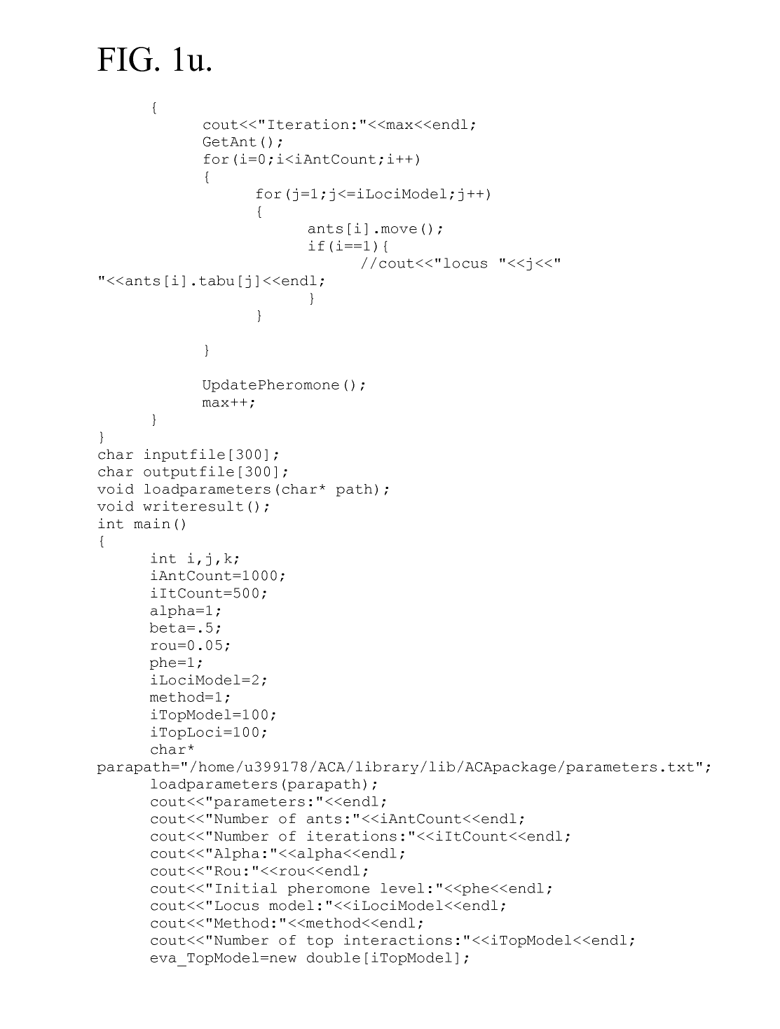

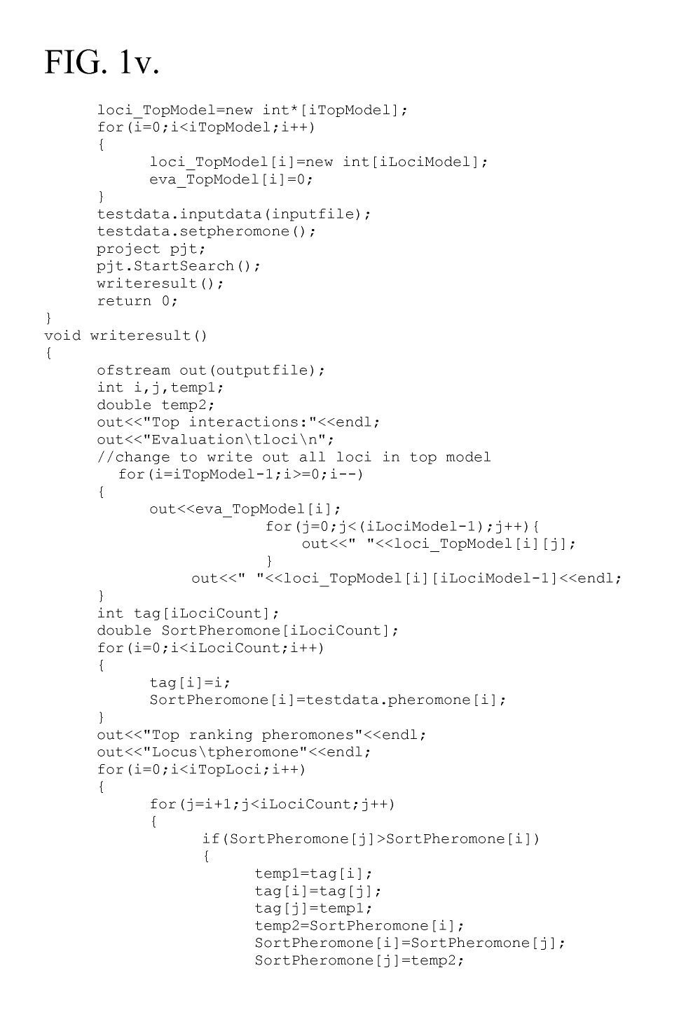

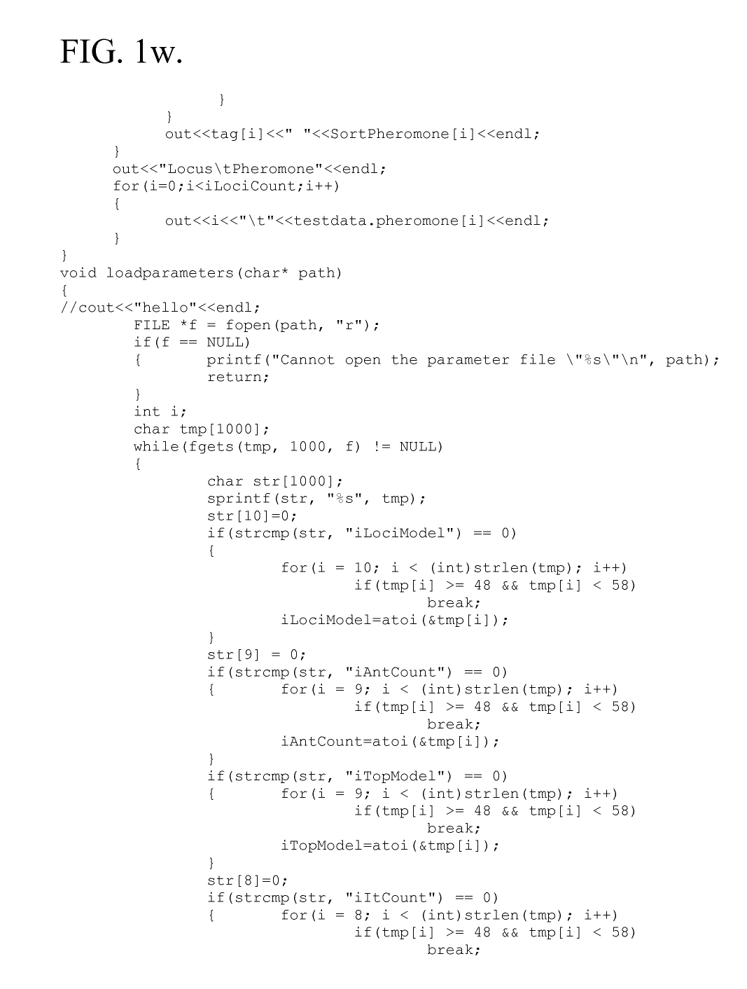

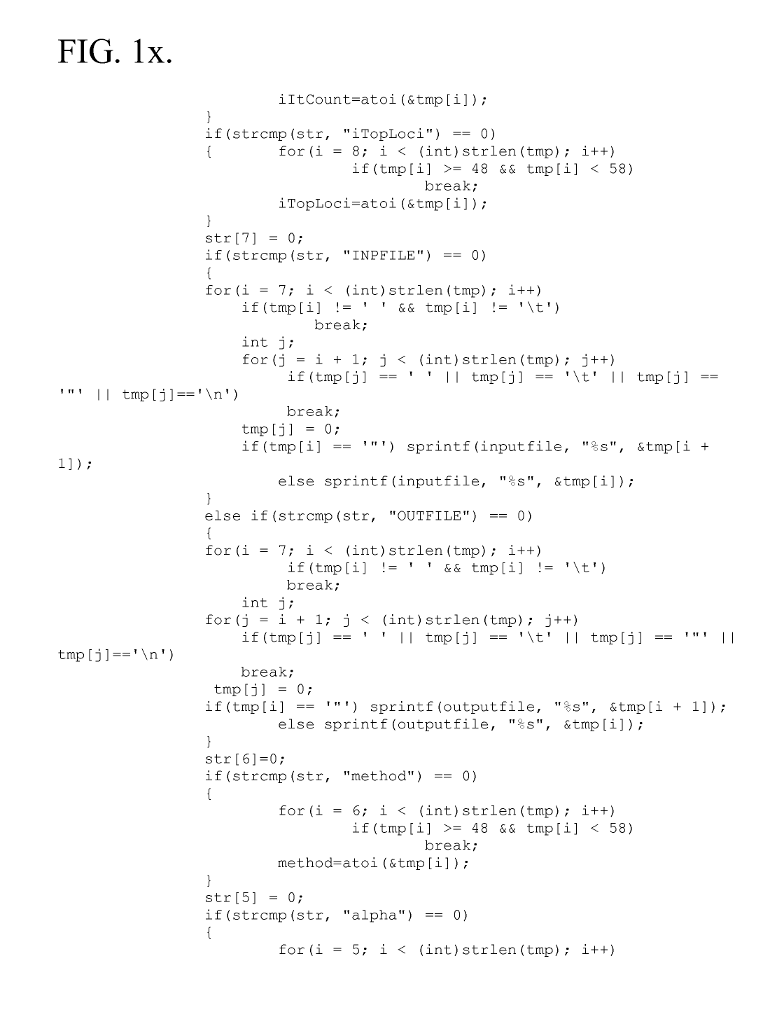

Following the update of pheromone levels according to Eq. 4, the PDF is updated according to Eq. 3, and the process is repeated until a convergence criteria is met, which in this example was a predefined number of iterations. As the PDF is updated, the selected features that perform better are sampled at higher likelihoods by subsequent artificial ants, which, in turn, deposit more “pheromone,” thus leading to a autocatalytic positive feedback system. FIG. 1 sets forth programming code used in this example to perform the above-described ant colony optimization procedure.

### Example 2

### Improved Performance of Ant Colony Optimization Systems

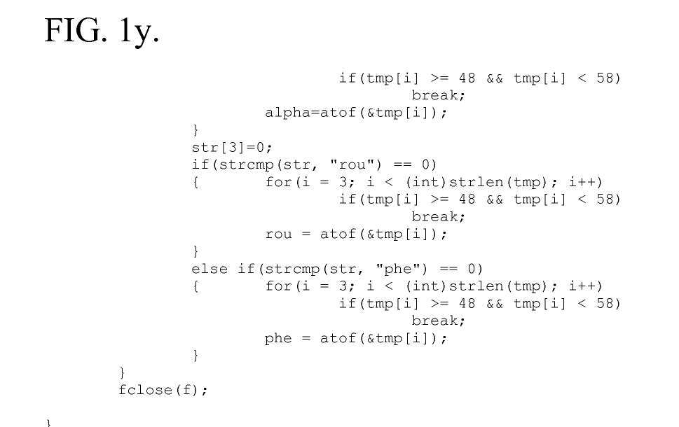

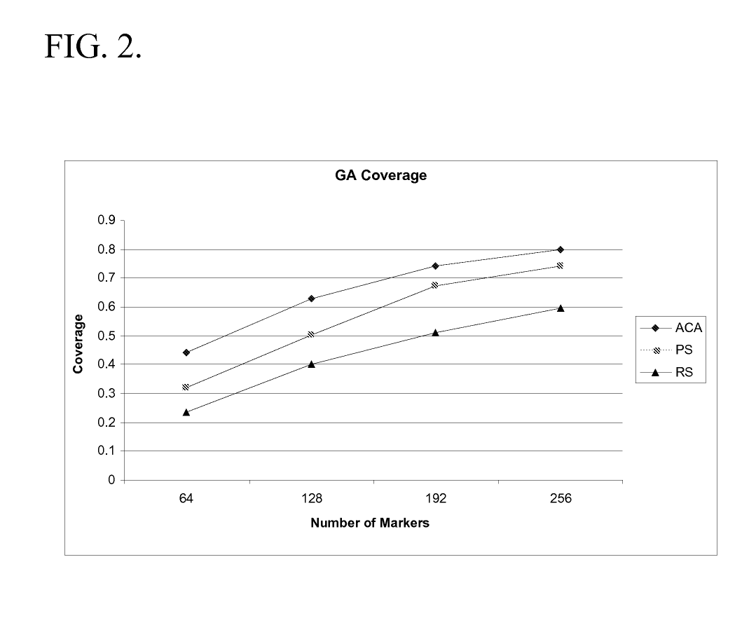

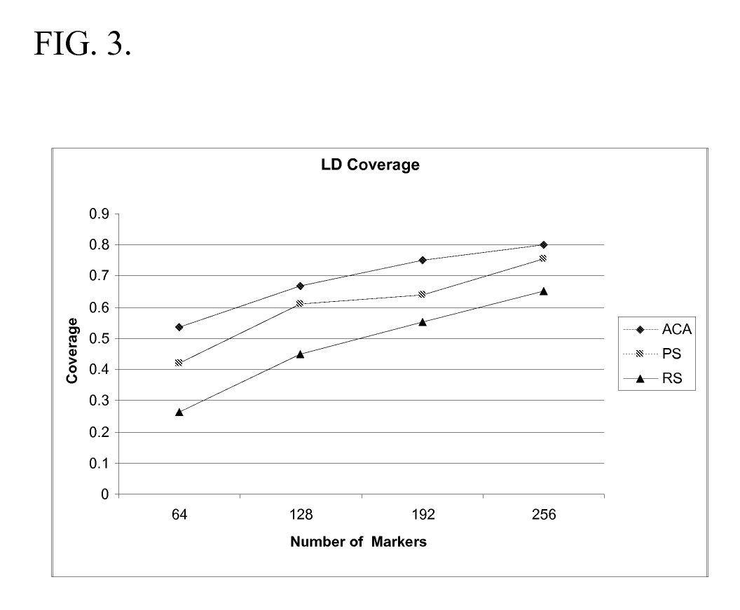

GA and LD coverage for marker panels selected using ACO, PS, and RS were determined. FIGS. 2-3. The ACO outperformed both PS and RS for all marker panel sizes tested, which was a clear indication that the adaptive sampling method of the ACO yields superior panel selections. At 256 markers, the panel selected by ACO for the GA and LD traits recovered 80% of the coverage obtained when using all available markers. Achieving this level of coverage using only 19% of the markers (256/1371 markers) is quite surprising and remarkable, particularly in view of the immense size of the sample space. Furthermore, the ACO converged to stable solutions in less than 5 minutes, indicating that larger sample spaces could be accommodated by the system.

### Example 3

### Optimized SNP Panel

The ACO was employed to select a 256 SNP panel for use in the TaqMan® OpenArray® SNP genotyping system. Given the importance of good LD coverage and the cost of large gaps in GA coverage, the criteria used for testing ACO performance were modified to place more weight on LD coverage and a higher penalty for large gaps in GA coverage. The new evaluation criteria calculated LD coverage using only the markers 25 cM upstream and downstream of inserts. For GA coverage, weights of markers covering more than 40 cM were set to 0, rather than 20 cM as previously described.

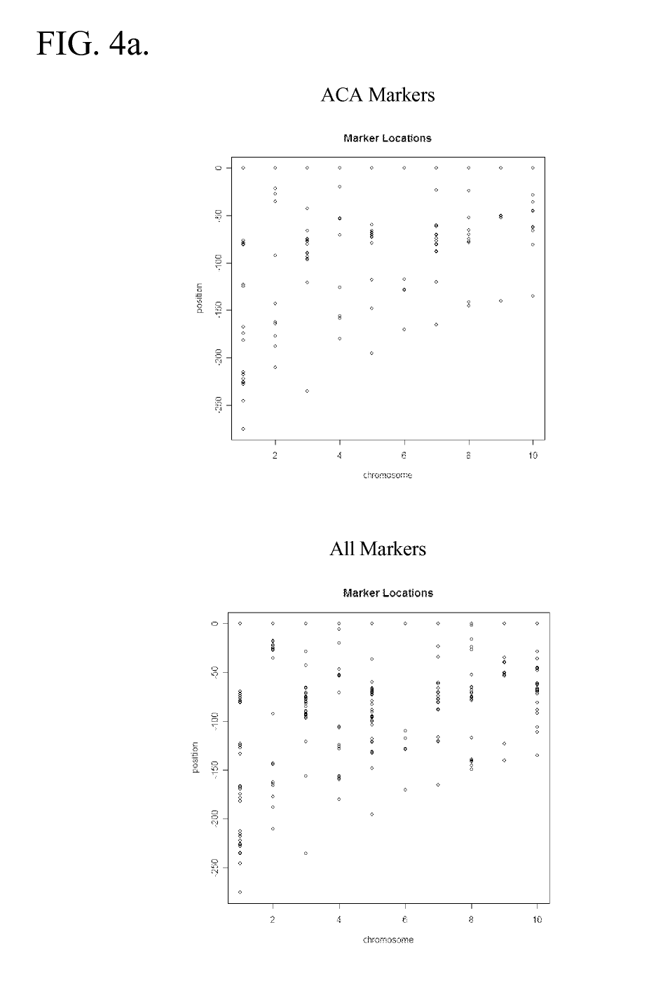

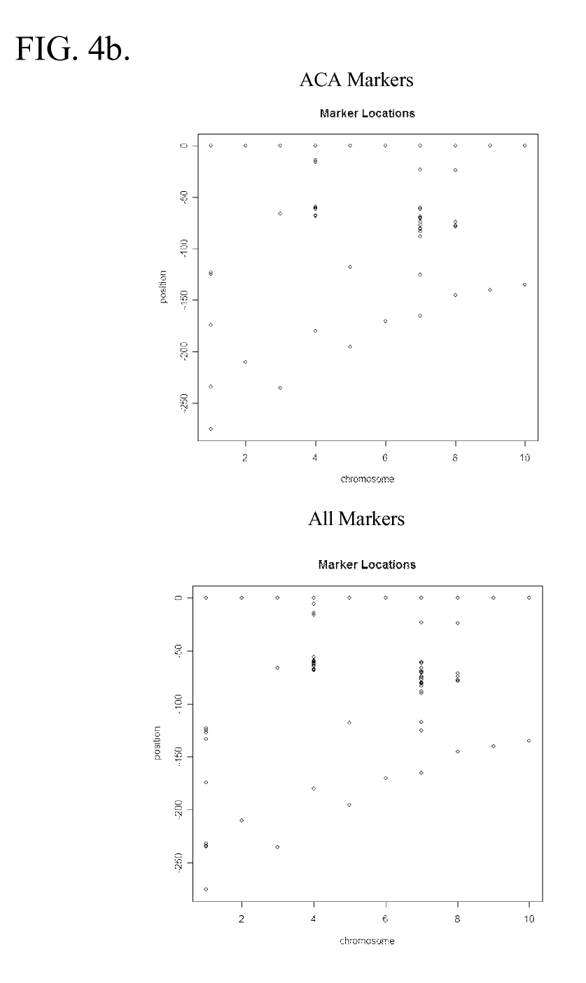

Using the new criteria, the ACA recovered 75% and 87% of the GA and LD coverage obtained using all available markers, respectively. Plots of the positions of informative markers can be found in FIG. 4 for two selected populations. While some large gaps in coverage are present, it can be seen that the gaps in the ACA panel correspond to gaps present when all informative markers are used. In general, the ACA panel yielded remarkably even coverage relative to the coverage obtained when using all available markers.

What may be claimed is:

1. A method of determining a set of biological markers for identification of a plant likely to comprise a trait of interest, the method comprising:
utilizing an ant colony optimization system to identify an optimized subset of plurality of genetic markers that is predictive of the trait of interest, wherein the optimized subset of the plurality of genetic markers is the set of biological markers for identification of a plant likely to comprise the trait of interest.

2. The method according to claim 1, further comprising:
providing an inbred plant that does not comprise the trait of interest, wherein the inbred plant comprises a genotype for the plurality of genetic markers;
crossing-a first donor plant comprising a genotype for the plurality of genetic markers with the inbred plant to produce a progeny plant, wherein the progeny plant comprises a genotype for the plurality of genetic markers, and determining whether the progeny plant comprises the trait of interest; and
providing a database comprising a plurality of genotypes for the plurality of genetic markers, wherein each genotype is the genotype of a progeny plant produced by crossing the inbred plant with an additional different donor plant.

3. The method according to claim 2, further comprising:
providing a genetic sample from the first donor plant; and
genotyping the first donor plant for the plurality of genetic markers.

4. The method according to claim 1, wherein utilizing an ant colony optimization system comprises:
defining a problem space comprised of discrete adjacent subsets of the plurality of genetic markers, and a plurality of agents, wherein the agents choose in successive time steps between the discrete adjacent subsets according to a probability density function that is updated over the successive time steps by a value determined by the genome (GA) and linkage drag (LD) coverage for the trait of interest provided by the chosen discrete adjacent subsets; and
allowing the agents to choose discrete adjacent subsets of the plurality of genetic markers over a predetermined number of successive time steps.

5. The method according to claim 4, wherein the discrete adjacent subset chosen in the last of the predetermined number of successive time steps is the set of biological markers for identification of a plant likely to comprise the trait of interest.

6. The method according to claim 1, wherein the genotype of the inbred plant for the plurality of genetic markers is determined by genotyping.

7. The method according to claim 1, wherein the genotype of the progeny plant for the plurality of genetic markers is determined by genotyping.

8. The method according to claim 1, wherein the genotype of an additional different donor plant is determined by genotyping.

9. The method according to claim 1, wherein the plurality of genetic markers comprise SNP markers.

10. The method according to claim 9, wherein the plurality of genetic markers consist of SNP markers.

11. The method according to claim 1, wherein the plant is selected from a group comprising maize, soybean, tobacco, carrot, canola, rapeseed, cotton, palm, peanut, Oryza sp., Arabidopsis sp., Ricinus sp., and sugarcane.

12. The method according to claim 1, wherein the plurality of genetic markers comprises at least about 1000 markers.

13. A set of markers determined by the method according to claim 1.

14. The set of markers of claim 13, wherein the set comprises less than about 300 markers.

15. A method of identifying a plant likely to comprise a trait of interest, the method comprising:
providing the set of markers of claim 13;
providing a genetic sample comprising nucleic acids from a plant; and
contacting the nucleic acids with probes that are specifically hybridizable to markers in the set of markers, wherein specific hybridization of the probes to the nucleic acids is indicative of the presence of the trait of interest in the plant.

16. The method according to claim 15, wherein the markers comprise SNP markers.

17. The method according to claim 15, wherein the plant is selected from a group comprising maize, soybean, tobacco, carrot, canola, rapeseed, cotton, palm, peanut, Oryza sp., Arabidopsis sp., Ricinus sp., and sugarcane.

18. A method of transferring a plant trait of interest, the method comprising:
providing the set of markers of claim 13;
providing a first parent plant comprising the trait of interest;
providing a second parent plant lacking the trait of interest;
analyzing the genomic DNA of the first and second parent plants with probes that are specifically hybridizable to markers in the set of markers, thereby determining the genotype of the first and second parent plants for the markers in the set of markers;
crossing the two parental plant genotypes to obtain a progeny population;
analyzing plants of the progeny population with probes that specifically hybridize to markers in the set of markers, thereby determining the genotype of the progeny plants for the markers in the set of markers;
backcrossing a progeny plant that comprises the same genotype as the first parent plant for the markers in the set of markers to the second parental genotype to produce a first backcross population; and
continuing with a backcrossing program until a final progeny plant is obtained that comprises any desired trait(s) exhibited by the second parent genotype and the same genotype as the first parent plant for the markers in the set of markers, thereby transferring the trait of interest.

19. The method according to claim 18, wherein the progeny of the first cross, or any subsequent backcross in the backcrossing program, is crossed to a third parent plant comprising a different genotype than either the first parent plant or second parent plant.

20. The method according to claim 18, wherein individual progeny obtained in each crossing and backcrossing step are genotyped for the markers in the set of markers.

21. The method according to claim 18, wherein the markers comprise SNP markers.

22. The method according to claim 18, wherein the plants are selected from a group comprising maize, soybean, tobacco, carrot, canola, rapeseed, cotton, palm, peanut, Oryza sp., Arabidopsis sp., Ricinus sp., and sugarcane.
